SEQUENCE LISTING (18 sequences)

SEQ ID NO: 1 - protein, 249 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 2 - DNA, 747 bp, synthetic construct
  source 1..747: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatggtagcagtggctggtacgtaccacactggttcgacccctggggccagggcaccctggtcaccgtctcctcaggcggcggcggcagcggcggcggcggcagcggcggcggcggcagcaattttatgctgactcagccccactctgtgtcggagtctccggggaagacggtaaccatctcctgcactcgcagcagtggcagcattgccagcaactatgtgcagtggtaccaacagcgcccgggcagttcccccaccactgtcatctatgaggataaccagagaccctctggggtccctgatcggttctctggctccatcgacagctcctccaattctgcctccctcaccatctctgggctgaagactgaggacgaggctgactactactgtcagtcttatgatggcagcaatcgttggatgttcggcggagggaccaagctgaccgtccta

SEQ ID NO: 3 - protein, 545 aa, synthetic construct
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVLGGGGSGGGGSGGGGSPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPV

SEQ ID NO: 4 - DNA, 1635 bp, synthetic construct
  source 1..1635: mol_type = other DNA
atggccctgcctgtgacagccctgctgctgcctctggctctgctgctgcatgccgctagacccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatggtagcagtggctggtacgtaccacactggttcgacccctggggccagggcaccctggtcaccgtctcctcaggcggcggcggcagcggcggcggcggcagcggcggcggcggcagcaattttatgctgactcagccccactctgtgtcggagtctccggggaagacggtaaccatctcctgcactcgcagcagtggcagcattgccagcaactatgtgcagtggtaccaacagcgcccgggcagttcccccaccactgtcatctatgaggataaccagagaccctctggggtccctgatcggttctctggctccatcgacagctcctccaattctgcctccctcaccatctctgggctgaagactgaggacgaggctgactactactgtcagtcttatgatggcagcaatcgttggatgttcggcggagggaccaagctgaccgtcctaggcggcggcggcagcggcggcggcggcagcggcggcggcggcagccccgaggagcccctggtggtgaaggtggaggagggcgacaacgccgtgctgcagtgcctgaagggcaccagcgacggccccacccagcagctgacctggagcagggagagccccctgaagcccttcctgaagctgagcctgggcctgcccggcctgggcatccacatgaggcccctggccatctggctgttcatcttcaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggacatgtgggtgatggagaccggcctgctgctgcccagggccaccgcccaggacgccggcaagtactactgccacaggggcaacctgaccatgagcttccacctggagatcaccgccaggcccgtg

SEQ ID NO: 5 - protein, 524 aa, synthetic construct
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 6 - DNA, 1572 bp, synthetic construct
  source 1..1572: mol_type = other DNA
cccgaggagcccctggtggtgaaggtggaggagggcgacaacgccgtgctgcagtgcctgaagggcaccagcgacggccccacccagcagctgacctggagcagggagagccccctgaagcccttcctgaagctgagcctgggcctgcccggcctgggcatccacatgaggcccctggccatctggctgttcatcttcaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggacatgtgggtgatggagaccggcctgctgctgcccagggccaccgcccaggacgccggcaagtactactgccacaggggcaacctgaccatgagcttccacctggagatcaccgccaggcccgtgggcggcggcggcagcggcggcggcggcagcggcggcggcggcagcgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatggtagcagtggctggtacgtaccacactggttcgacccctggggccagggcaccctggtcaccgtctcctcaggcggcggcggcagcggcggcggcggcagcggcggcggcggcagcaattttatgctgactcagccccactctgtgtcggagtctccggggaagacggtaaccatctcctgcactcgcagcagtggcagcattgccagcaactatgtgcagtggtaccaacagcgcccgggcagttcccccaccactgtcatctatgaggataaccagagaccctctggggtccctgatcggttctctggctccatcgacagctcctccaattctgcctccctcaccatctctgggctgaagactgaggacgaggctgactactactgtcagtcttatgatggcagcaatcgttggatgttcggcggagggaccaagctgaccgtccta

SEQ ID NO: 7 - protein, 9 aa, synthetic construct
EQKLISEDL

SEQ ID NO: 8 - DNA, 30 bp, synthetic construct
  source 1..30: mol_type = other DNA
gagcaaaagctcatttctgaagaggacttg

SEQ ID NO: 9 - protein, 6 aa, synthetic construct
HHHHHH

SEQ ID NO: 10 - DNA, 18 bp, synthetic construct
  source 1..18: mol_type = other DNA
caccatcaccatcaccat

SEQ ID NO: 11 - protein, 20 aa, synthetic construct
LEAGCKNFFPRSFTSCGSLE

SEQ ID NO: 12 - DNA, 60 bp, synthetic construct
  source 1..60: mol_type = other DNA
ytngargcnggntgyaaraayttyttyccnmgnwsnttyacnwsntgyggnwsnytngar

SEQ ID NO: 13 - protein, 581 aa, synthetic construct
MALPVTALLLPLALLLHAARPQVQLQESGPGLVKPSETLSLTCTVSINYTGEPTYSQSVSNVVWYWIRQPPGKGLEWIGYTYYPTVNENRSQSVSNVFRVTISVDTSKNQFSLKLSSVTAADTAVYYCARSSYPGYSQPTRSYPSSWGQGTLVTVSSGGGGSGGGGSGGGGSQSVLTQPPSVSGAPGQRVTISCSGSSSNIGSNYVSWYQQLPGTAPKLLIYDNNQRPSGVPDRFSGSKSGTSASLAITGLQSEDEADYYCYINTGYDGLYYNSMDFGGGTKLTVLGGGGGSGGGGSGGGGSPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVAAAEQKLISEEDLHHHHHH

SEQ ID NO: 14 - DNA, 1758 bp, synthetic construct
  source 1..1758: mol_type = other DNA
ctcgagatggccctgcctgtgacagccctgctgctgcctctggctctgctgctgcatgccgctagaccccaggtgcagctgcaggagagcggccccggcctggtgaagcccagcgagaccctgagcctgacctgcaccgtgagcatcaactacaccggcgagcccacctacagccagagcgtgagcaacgtggtgtggtactggatcagacagccccccggcaagggcctggagtggatcggctacacctactaccccaccgtgaacgagaacagaagccagagcgtgagcaacgtgttcagagtgaccatcagcgtggacaccagcaagaaccagttcagcctgaagctgagcagcgtgaccgccgccgacaccgccgtgtactactgcgccagaagcagctaccccggctacagccagcccaccagaagctaccccagcagctggggccagggcaccctggtgaccgtgagcagcggcggcggcggcagcggcggcggcggcagcggcggcggcggcagccagagcgtgctgacccagccccccagcgtgagcggcgcccccggccagagagtgaccatcagctgcagcggcagcagcagcaacatcggcagcaactacgtgagctggtaccagcagctgcccggcaccgcccccaagctgctgatctacgacaacaaccagagacccagcggcgtgcccgacagattcagcggcagcaagagcggcaccagcgccagcctggccatcaccggcctgcagagcgaggacgaggccgactactactgctacatcaacaccggctacgacggcctgtactacaacagcatggacttcggcggcggcaccaagctgaccgtgctgggcggcggcggcggcagcggcggcggcggcagcggcggcggcggcagccccgaggagcccctggtggtgaaggtggaggagggcgacaacgccgtgctgcagtgcctgaagggcaccagcgacggccccacccagcagctgacctggagcagggagagccccctgaagcccttcctgaagctgagcctgggcctgcccggcctgggcatccacatgaggcccctggccatctggctgttcatcttcaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggacatgtgggtgatggagaccggcctgctgctgcccagggccaccgcccaggacgccggcaagtactactgccacaggggcaacctgaccatgagcttccacctggagatcaccgccaggcccgtggccgccgccgagcaaaagctcatttctgaagaggacttgcaccatcaccatcaccattgaatcgat

SEQ ID NO: 15 - protein, 15 aa, synthetic construct
GGGGGGGGGGGGSSS

SEQ ID NO: 16 - protein, 4 aa, synthetic construct
GGGS

SEQ ID NO: 17 - DNA, 1758 bp, synthetic construct
  source 1..1758: mol_type = other DNA
atcgattcaatggtgatggtgatggtgcaagtcctcttcagaaatgagcttttgctcggcggcggccacgggcctggcggtgatctccaggtggaagctcatggtcaggttgcccctgtggcagtagtacttgccggcgtcctgggcggtggccctgggcagcagcaggccggtctccatcacccacatgtccctggcgggcctgtcgtccttcagctccaggctcagcaggctcttggggcccttggggtgcacgtgggtccagctcagggggcccctgctcacgctgtcggggggcacgccgcagctcagccacagggtgctgccgggggccatggtcaggtcctggctcaggctctggttcaggctgtccctggggggcaggcaggggggctcgccctcccagatctcgggcctgtccttggcccacacgtacagcttggggctcatcagcttgccgctggggctgctggggccctcgctgctcctgttcttcaggccgcagcccaggccgcccaggtcgctcacgttccacctgaacagctcgccgctgccctccacgttcacggtccagccgggctgccaggccttctcgctgggggggccgggctggcacaggtagaagccgcccatctgctggctcacgttgaagatgaacagccagatggccaggggcctcatgtggatgcccaggccgggcaggcccaggctcagcttcaggaagggcttcagggggctctccctgctccaggtcagctgctgggtggggccgtcgctggtgcccttcaggcactgcagcacggcgttgtcgccctcctccaccttcaccaccaggggctcctcggggctgccgccgccgccgctgccgccgccgccgctgccgccgccgccgcccagcacggtcagcttggtgccgccgccgaagtccatgctgttgtagtacaggccgtcgtagccggtgttgatgtagcagtagtagtcggcctcgtcctcgctctgcaggccggtgatggccaggctggcgctggtgccgctcttgctgccgctgaatctgtcgggcacgccgctgggtctctggttgttgtcgtagatcagcagcttgggggcggtgccgggcagctgctggtaccagctcacgtagttgctgccgatgttgctgctgctgccgctgcagctgatggtcactctctggccgggggcgccgctcacgctggggggctgggtcagcacgctctggctgccgccgccgccgctgccgccgccgccgctgccgccgccgccgctgctcacggtcaccagggtgccctggccccagctgctggggtagcttctggtgggctggctgtagccggggtagctgcttctggcgcagtagtacacggcggtgtcggcggcggtcacgctgctcagcttcaggctgaactggttcttgctggtgtccacgctgatggtcactctgaacacgttgctcacgctctggcttctgttctcgttcacggtggggtagtaggtgtagccgatccactccaggcccttgccggggggctgtctgatccagtaccacaccacgttgctcacgctctggctgtaggtgggctcgccggtgtagttgatgctcacggtgcaggtcaggctcagggtctcgctgggcttcaccaggccggggccgctctcctgcagctgcacctggggtctagcggcatgcagcagcagagccagaggcagcagcagggctgtcacaggcagggccatctcgag

SEQ ID NO: 18 - protein, 585 aa, synthetic construct
LEMALPVTALLLPLALLLHAARPQVQLQESGPGLVKPSETLSLTCTVSINYTGEPTYSQSVSNVVWYWIRQPPGKGLEWIGYTYYPTVNENRSQSVSNVFRVTISVDTSKNQFSLKLSSVTAADTAVYYCARSSYPGYSQPTRSYPSSWGQGTLVTVSSGGGGSGGGGSGGGGSQSVLTQPPSVSGAPGQRVTISCSGSSSNIGSNYVSWYQQLPGTAPKLLIYDNNQRPSGVPDRFSGSKSGTSASLAITGLQSEDEADYYCYINTGYDGLYYNSMDFGGGTKLTVLGGGGGSGGGGSGGGGSPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVAAAEQKLISEEDLHHHHHHID

(19) United States
(12) Patent Application Publication
(10) Pub. No.: US 2024/0060043 A1
(43) Pub. Date: Feb. 22, 2024

(54) METHODS AND COMPOSITIONS COMPRISING FUSION PROTEINS FOR THE IDENTIFICATION OF IMMUNOTHERAPY CELLS

(71) Applicants: Villanova University, Villanova, PA (US); Lankenau Institute for Medical Research, Wynnewood, PA (US)

(72) Inventors: William Joseph Kelly, III, Schwenksville, PA (US); Justin Thomas Fisher, Indian Trail, NC (US); Scott Kendall Dessain, Wynnewood, PA (US)

(73) Assignees: Villanova University, Villanova, PA (US); Lankenau Institute for Medical Research, Wynnewood, PA (US)

(21) Appl. No.: 18/357,452

(22) Filed: Jul. 24, 2023

Related U.S. Application Data
(60) Provisional application No. 63/391,566, filed on Jul. 22, 2022.

(51) Int. Cl. C12N 5/0783; C07K 16/28; C07K 14/57; B01D 15/38; C07K 14/525

(52) U.S. Cl. CPC C12N 5/0636; C07K 16/2803; C07K 14/57; B01D 15/3809; C07K 14/525; C07K 2319/00; C07K 2317/622

(57) ABSTRACT

Fusion proteins, comprising at least one single chain antibody fragment (scFV), and a cancer antigen or marker, or a fragment thereof are provided. The scFV is capable of selectively binding to a cytokine released by chimeric antigen receptor (CAR) expressing cell such as interferon gamma (IFN-γ) or tumor necrosis factor alpha (TNF-α). The cancer antigen or marker may comprise the extracellular domain of CD19. Methods for isolating or purifying CAR expressing cells using fusion proteins are provided.

### PRIORITY CLAIM AND CROSS-REFERENCE

This application claims priority to U.S. Patent Application No. 63/391,566 filed on Jul. 22, 2022. The foregoing application, and all documents cited therein, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically and is hereby incorporated by reference in its entirety. Said XML copy, was created Jul. 24, 2023, is named E8144-00073.xml and is 26,658 bytes in size.

### FIELD OF THE INVENTION

The disclosure relates to immunology and protein engineering generally. More particularly, the disclosed subject matter relates to novel fusion proteins, methods of using such fusion proteins, and methods of producing, isolating or purifying activated immunotherapy cells such as T cells including human chimeric antigen receptor (CAR) T cells, for use in immunotherapies.

### BACKGROUND OF THE INVENTION

CAR T cell therapy has revolutionized treatment of blood cancers such as leukemias and lymphomas. Long-term results of this immunotherapy have shown increased rates of remission for patients and have a promising future for targeting new cancers. This type of immunotherapy is achieved by genetically modifying a patient's T lymphocytes so that they are equipped with artificial chimeric antigen receptors that target cancer cell surface antigens. As effective as immunotherapy can be, significant improvement could be achieved if patients receive more potent doses of CAR T cells with high efficacy. Currently, only the efficacy of a polyclonal CAR T cell population is determined. This is done with in vitro assays involving CAR T cell populations exposed to tumor-associated antigens (TAA) on beads/plates. After a certain time of exposure, the supernatant samples are tested for an increase in cytokine levels. Other assays involve a population of TAA-presenting cells exposed to a population of CAR T cells, where either supernatant is analyzed or the number of TAA-presenting cells killed by CAR T cells is determined with flow cytometry.

The potential of CAR T cell therapy to address a myriad of physiological conditions is significant: this approach has been utilized over the previous two decades to redirect T cell specificity in immunotherapy research and has been successful in the translation to clinical applications. In 2017 the FDA approved Kymriah™ (Tisagenlecleucel) for the treatment of patients with relapsed/refractory (r/r) B cell precursor acute lymphoblastic leukemia [1] or diffuse large B cell lymphoma (DLBCL) [2] and Yescarta™ (Axicabtagene Ciloleucel) for the treatment of patients with r/r DLBCL and primary mediastinal B cell lymphoma (PMBCL) [3]. These two groundbreaking CAR T cell therapies targeted CD19, the tumor marker expressed in most of the B lineage hematological malignancies, and have led to a rapid advance in the growth of CAR-T therapy development. However, despite this success there has been a large variation in responses and unpredictable toxicity in patients—which is partially attributed to inter-patient heterogeneity of CAR-T product infusion [4-6]. Thus, there is a need for allogeneic products to be developed that, due to economies of scale, will need to eventually replace these costly and variable autologous therapies [7]. There are currently over 650 T cell immunotherapy products in clinical trials, including allogeneic products such as CYAD-101™ by Celyad and ALLO-501 by Allogene [8-10].

Clinically, the long-term efficacy of CAR-T cells is linked to their proliferative capacity and their long-term anti-tumor activity, as well as the cellular composition of the final CAR-T product [6]. Therefore, at least one approach for improving clinical success of CAR-T cell therapies is the selection of specific CAR-T subpopulations and subsets. This approach is supported by the finding that above a certain threshold, the absolute number of transfused CAR-T cells does not directly correlate with in vivo expansion and clinical success, highlighting the importance of cellular composition in the efficacy of the transfused CAR-T cells [11]. Further, as CAR-T cell therapy continues to develop, so will regulatory requirements by the FDA and other such organizations worldwide. Therefore, future therapies will likely need to demonstrate an increased potency for reproducibility. Selecting defined subsets of T cells prior to transduction and formulating therapeutic CAR-T products of uniform composition, provides a more reproducible potency, thus aids in determining potential correlations in cell dose and efficacy and toxicity during clinical trials [12].

One approach for producing a uniform composition is a CAR product with uniform CAR expression levels. The most common method for CAR gene transduction is using lentiviral vectors carrying the CAR transgene. Lentiviral vectors provide a stable CAR expression via integration into the host genome; however, random integration may result in variegated CAR expression and ultimately lead to a reduced in vivo persistence of CAR-T cells [13]. Non-viral gene editing, such as CRISPR/Cas9 site-specific integration results in a uniform CAR expression however CRISPR/Cas9 and other non-viral methods are currently not utilized in clinical settings due to a much lower transduction efficiency [14]. Thus, there is a need for utilizing a process-step for identifying a uniform CAR expression profile.

Single-cell cytokine expression studies [5,15] of CAR-T cells has also shown that the levels of cytokine expression are highly variable. It seems likely that expression of these cytokines by CAR-T cells reflects an activated state that will correlate with cell longevity and anti-tumor activity in patients. Recent single-cell multiomic profiling of CD19 CAR-T infusion products from acute lymphoblastic leukemia (ALL) patients revealed that stimulated populations had upregulation of functional effectors, including colony-stimulating factor, INF-y and granzyme B [16]. If “activated” CAR-T cells (as determined by high levels of cytokine expression) give improved efficacy of CAR-T treatments, then future CAR-T cell manufacturing processes need to efficiently create and deliver a product enriched in activated cells. Current technologies do not allow CAR-T cell cytokine expression profiles to be evaluated prior to infusing CAR-T cells into the patients.

Cytokine production by activated CAR-T cells not only plays a role in mediating tumor growth, it can also enhance CAR-T anti-tumor efficacy [17]. Specifically, IFN-7, which is released in large amounts in activated CD8 T cells, is a key moderator of cell-mediated immunity and has a variety of pro-inflammatory functions in CD8 cells. IFN-7 binds to CD8 cells via the IFN-γR and can induce IL-12 receptor expression and can enhance the ability of cytotoxic CD8 T cells to kill via Fas/FasL axis in absence of perforin [18]. Further, CD8 T cells which do not express the IFN-γR have been shown to be unable to lyse antigen-expressing cells, indicating the cytolytic function of these CD8 T cells is depending upon their IFN-γR expression.

Additionally, one study characterizing the therapeutic effects of novel anti-CD38 CAR-T cells found that following stimulation with CD38+ tumor cells, the CAR-T cells secreted high levels of IFN-7 and subsequently promoted tumor cell apoptosis in vitro. Further in vivo experiments demonstrated the anti-CD38 CAR-T cells could be activated to secrete IFN-7 and eliminate tumors [19]. CRISPR knockout screens in glioblastoma's, which CAR-T cells have limited efficacy, showed that loss of genes in the IFN-γR pathway reduced the killing efficiency of CAR-T cells both in vitro and in vivo [20]. The loss of IFN-γR1 specifically led to reduced overall binding duration and avidity of CAR-T cells [20]. Interestingly, CD8 effector cells have been shown to vary in their sensitivity to IFN-γ, suggesting different populations of varying cytolytic activity exist, further suggesting that IFN-7 can be used as an efficacy marker to isolate cells on their various degree of efficacy [21]. Due to the influence of IFN-7 on both cytotoxic CD8 and CAR-T cells' cytotoxicity and activation, this cytokine is an interesting target for efficacy marker.

There have been attempts to develop polyclonal cell culture methods where mAbs are associated with the cells that secrete them through a variety of methods, such as trapping secreted mAbs in a secretion capture report web (SCRW), gel microdroplets, or semi-solid medium [22-24]. While these methods are useful for isolating hybridomas, the utility in isolating specific CAR-T populations is questionable. Other methods have been designed to directly attach mAbs to the hybridomas that secrete them by using a secondary antibody that adheres to the hybridoma surface and capture secreted mAbs however these methods do not prevent mAbs from binding to nearby cells that did not secrete them and require testing the hybridoma mAbs in assays separate from the hybridoma cultures.

What is needed is an improved methodology for identifying and selecting immunotherapy cells having particular characteristics. What is also needed are tools and protocols that allow for the characterization of immunotherapy cells, such as those that allow for the analysis of single-cell cytokine secretion by such cells, and also enable the subsequent identification and isolation of specific cytokine secreting cell populations.

### SUMMARY OF THE INVENTION

Provided herein are improved methodologies and techniques for identifying and selecting immunotherapy cells having particular characteristics comprising the use of novel fusion proteins. The fusion proteins of the invention uniquely allow for the characterization of immunotherapy cells, by mechanisms such as by identifying cytokine secretion by such cells, and also enable the subsequent isolation of specific cytokine-secreting cell populations.

The present disclosure provides novel fusion proteins, methods of making the same, and methods of using the same.

The present disclosure provides methods for isolating or purifying CAR expressing cells with high efficiency.

The inventors herein have developed novel fusion proteins comprising components that recognize both immunotherapy cells and cytokines released by such cells. In an embodiment, the fusion proteins are engineered to bind to CAR cells, including, but not limited to for example, tumor antigens or cancer biomarkers such as CD19 components (B cell marker). In an embodiment, the fusion proteins are engineered to recognize cytokines released by immunotherapy cells, including, but not limited to, interferon gamma (IFN-γ) or tumor necrosis factor alpha (TNF-α). Such fusion proteins comprise at least one single chain antibody fragment (scFV), and a component that binds the immunotherapy cell, such as the extracellular domain of CD19, or a fragment thereof. In an embodiment, the scFV is capable of selectively capturing secreted cytokines such as IFN-γ or TNF-α, and the fusion protein is configured to isolate or purify the CAR expressing cell.

In an embodiment, provided herein are IFN-γ scFv:CD19 extracellular domain (ECD) fusion proteins which when incubated with anti-CD19 CAR expressing cells will facilitate membrane capture of secreted IFN-γ. Use of this fusion protein allows for the analysis of single-cell cytokine secretion and the subsequent identification and isolation of specific IFN-γ secreting populations.

In an embodiment, provided herein are TNF-α scFv:CD19 extracellular domain (ECD) fusion proteins which when incubated with anti-CD19 CAR expressing cells will facilitate membrane capture of secreted TNF-α. Use of this fusion protein allows for the analysis of single-cell cytokine secretion and the subsequent identification and isolation of specific TNF-α secreting populations.

In an embodiment, the fusion proteins described herein selectively bind specific cytokines wherein the cytokines may comprise interferons, interferon gamma (IFN-γ), TNF-α, interleukins, IL1 IL2 IL6, IL10, IL12, I15, IL17, IL18, IL21 IL35, growth factors, CSF, chemokines, lymphokines and monokines. The immunotherapy cell may comprise T cells.

In certain embodiments, the fusion proteins further comprise at least one (or more) linkers between scFV and the extracellular domain of CD19. The scFV in the fusion protein may also include different regions and linkers therebetween.

In certain embodiments, the scFV comprises the amino acid sequence of SEQ ID NO:1 or the nucleotide sequence of SEQ ID NO:2. For example, the fusion protein comprises the amino acid sequence of SEQ ID NO: 3 or SEQ ID NO: 5. Their corresponding nucleotide sequence is SEQ ID NO:4 or SEQ ID NO:6, respectively. The fusion protein comprising the amino acid sequence of SEQ ID NO: 3 or SEQ ID NO: 5 may be used to selectively identify CAR-T cells that secrete IFN-7.

In certain embodiments, the scFV comprises the amino acid sequence of SEQ ID NO: 13 or the nucleotide sequence of SEQ ID NO: 14. The fusion protein comprising the amino acid sequence of SEQ ID NO: 13 may be used to selectively identify CAR-T cells that secrete TNF-α.

In another aspect, the present disclosure provides methods for producing fusion proteins, comprising at least one expressing step as described herein.

In another aspect, the present disclosure provides methods for using the novel fusion proteins of the invention for identifying, isolating or purifying immunotherapy cells such as CAR expressing cells, including CAR-T cells. In an embodiment for the method of isolating or purifying a CAR expressing cell, the scFV of the fusion protein binds with secreted IFN-γ from a chimeric antigen receptor (CAR) expressing cell, and the extracellular domain of CD19 binds with an anti-CD19 chimeric antigen receptor (CAR). In another embodiment for the method of isolating or purifying a CAR expressing cell, the scFV of the fusion protein binds with secreted TNF-α from a chimeric antigen receptor (CAR) expressing cell, and the extracellular domain of CD19 binds with an anti-CD19 chimeric antigen receptor (CAR). The method for isolation or purification of the CAR expressing cell can be performed in a bed reaction packed with beads made of a polymer. The polymer may be cross-linked and porous. The polymer beads may be configured to capture the fusion protein and subsequent steps may be employed to dissociate and isolate/collect the immunotherapy cell from the fusion protein. The method includes the steps as described herein.

The technology provided in the present disclosure can be used to identify CAR-T-cells, such as CD8+ CAR-T-cells, and select those with cytokine production, such as IFN-γ or TNF-α production. This allows for the selection of only the most highly efficacious T-cells to be used for treating patients with diseases or conditions such as cancer.

### BRIEF DESCRIPTION OF THE DRAWINGS

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 illustrates two exemplary fusion protein in accordance with certain embodiments: FIG. 1A. Fusion protein with the IFN-γ scFv on the N-terminal side, and a C-terminal CD19 ECD; and FIG. 1B. Fusion Protein with the CD19 ECD on the N-Terminal side and the IFN-γ on the C-terminal side. The CMV promoter, leader sequence (S), V_{H} and V_{L} regions of anti-IFN-γ scFv, flexible peptide linker (Link), CD19 Extracellular Domain (ECD), Myc-tag, and 6×-His tagged sequence are shown.

FIG. 2 illustrates modeled IFN-γ:CD19 Fusion Protein bound to IFN-γ, in which homology modelling was used to develop a 3D image of the structure of the fusion protein. FIG. 2A. Identification of the IFN-γ scFv and CD19 ECD domains. FIG. 2B. Structure of the fusion protein with IFN-γ bound to the IFN-γ scFv domain (as shown in the dotted circle).

FIG. 3 illustrates expression vector for the production of IFN-γ:CD19 Fusion Protein in accordance with certain embodiments.

FIG. 4 illustrates that biotinylated goat anti-rabbit IgG binds streptavidin coated macrobead in accordance with certain embodiments.

FIG. 5 illustrates that primary goat anti-rabbit IgG captures secondary rabbit Anti-interferon gamma (IFN-γ) IgG in accordance with certain embodiments.

FIG. 6 illustrates that rabbit anti-IFN-γ IgG captures secreted IFN-γ in accordance with certain embodiments.

FIG. 7 illustrates that IFN-γ:CD19 fusion protein binds captured IFN-γ stabilizing immune complex in accordance with certain embodiments.

FIG. 8 illustrates that T-Cell secreting IFN-γ expresses anti-CD19 CAR, which binds CD19 on fusion protein in accordance with some embodiments.

FIG. 9 illustrates the construction and size characterization of the novel fusion proteins of the invention: FIG. 9A Diagram of the viral vector construct used to transduce the CD19 ECD: IFN-γ scFv fusion genes; FIG. 9B Reduced, denaturing protein electrophoresis by SDS PAGE of fusion protein products either treated with NEB deglycosylation kit or native with Lanes (1) Deglycosylated IFN-γ scFv: CD19 ECD Fusion Protein (2) Native IFN-γ scFv:CD19 ECD Fusion Protein (3) Native CD19 ECD: IFN-γ scFv Fusion Protein (4) Deglycosylated CD19 ECD: IFN-γ scFv Fusion Protein (5) Fetuin Control (6) Deglycosylated Fetuin Control. Box surrounding bands on Lanes 1-4 show expected size band for each fusion protein orientation.

FIG. 10 provides data related to recombinant IFN-γ binding evaluation of fusion proteins. FIG. 10A Results of a comparative sandwich ELISA using each fusion protein orientation binding to soluble IFN-γ; FIG. 10B Results of a competitive binding assay using the IFN-γ scFv: CD19 ECD fusion protein with or without a competitor rabbit anti-human IFN-γ polyclonal mAb; FIG. 10C Results of a competitive binding assay using the CD19 ECD: IFN-γ scFv fusion protein with or without a competitor rabbit anti-human IFN-γ 281 polyclonal mAb.

FIG. 11 illustrates the results of a comparative sandwich ELISA using each fusion protein orientation binding to a FMC63-Fc protein.

FIG. 12 illustrates the results of IFN-γ scFv: CD19 ECD Binding to anti-CD19 CAR Expressing HEK293T (CAR-H) after gating for CAR+. FIG. 12A Representative histogram demonstrating binding of 136 ng/L fusion protein while binding at room temperature; FIG. 12B Representative histogram demonstrating binding of 13.6 ng/L fusion protein while binding at room temperature; FIG. 12C Representative histogram demonstrating binding of 136 ng/L fusion protein while binding at 37° C.; FIG. 12D Representative histogram demonstrating binding of 13.6 ng/L fusion protein while binding at 37° C.

FIG. 13 illustrates in vitro binding of IFN-γ scFv: CD19 ECD fusion protein to anti-CD19 CAR-T Cells. Mean Fluorescent Intensity (MFI) evaluation following gating for CAR+ (via GFP) and Fusion Protein+ (via PerCp-Streptavidin).

FIGS. 14A-14C illustrate data related to dynamic binding interaction evaluation between IFN-γ scFv: CD19 ECD Fusion Protein and anti-CD19 CAR-T Cells. Flow Cytometry Scatter Plot demonstrating difference in FSC vs SSC in Fixed (FIG. 14A) vs Non-Fixed (FIG. 14B) anti-CD19 CAR-T cells following incubation with fusion protein (FIG. 14C). Representative histogram depicting differences in IFN-γ scFv: CD19 ECD fusion protein binding to CAR-T cells when either first incubated with the PerCp-Streptavidin (Grey) or first incubated with the CAR-T cells (white).

FIGS. 15A-15D illustrate IFN-γ scFv:CD19 ECD Fusion Protein CAR-T Selection (FIG. 15A). FSC vs SSC Scatter Plot following binding of fusion protein to CAR-T cells with three colored gates demonstrating three levels of ascending FSC and SSC(FIG. 15B). CAR-expression evaluation with three colored gates from Panel A demonstrating increased FSC and SSC corresponds to increased CAR expression (FIG. 15C). Representative histogram from fusion protein binding with K-means clustering to identify three clusters of varying fusion protein binding levels (FIG. 15D). Flow cytometry scatter plot following binding of fusion protein to CAR-T cells with three colors identifying the three fusion protein binding level clusters.

FIG. 16 illustrates a cloned portion of pTwist-CMV-BetaGlobin-WPRE-Neo plasmid, including Myc-tag, 6×-His tag, and TNF-α scFv and CD19 extracellular domain linked via a flexible peptide (Gly4Ser)3 (SEQ ID NO:15) linker.

FIG. 17 illustrates a gel with supernatant samples after 2 and 3 days of HEK298 incubation.

FIG. 18 illustrates the average moles of biotin per mole of fusion protein. The first result analyzed biotinylated fusion protein that had not been filtered by a Zeba spin column, showing that nitrogenous bases interfered with biotin binding to fusion protein. The others had nitrogenous bases removed in the Zeba spin column and showed higher levels of biotin per molecule of fusion protein. Absorbances for calculation were read at 500 nm.

FIG. 19 illustrates a sandwich ELISA performed with TNF-α serial dilution 100-6.125 pg/mL. Biotinylated fusion protein with 2.1 moles bioitin per mole of fusion protein. 2.1 molecules fusion protein per molecule of biotin. Absorbance at 450 nm.

FIG. 20 illustrates a TNF-α binding ELISA where 500 ng/well of TNF-α was added to the bottom of the well replacing the capture antibody. Biotinylated CD19:TNF-α fusion protein was added in 10× dilutions between 500 ng/well and 0.05 ng/well. 2.1 molecules fusion protein per molecule of biotin. Absorbance 450 nm.

FIG. 21 illustrates competition ELISA results, where half of the wells contained 500 ng/well anti-TNF-α mAb before the addition of 500 ng/well biotinylated CD19:TNF-α fusion protein to all wells. 500 ng/well of TNF-α was added to the bottom of the well replacing the capture antibody. 3.3 molecules fusion protein per molecule of biotin. Absorbance 450 nm.

FIG. 22 ELISA results using fusion protein produced in HEK293 cells 6 months earlier. Capture antibody replaced by 500 ng/well of FMC63. TNF-α:CD19 biotinylated fusion protein was added in 500, 0.5, or 0 ng/well. 2.1 molecules fusion protein per molecule of biotin. Absorbance 450 nm.

FIG. 23 illustrates ELISA results using fusion protein produced in HEK293 cells the week of the ELISA run. Capture antibody replaced by 500 ng/well of FMC63. Biotinylated TNF-α:CD19 fusion protein was added in 500, 0.5, or 0 ng/well. 3.3 molecules fusion protein per molecule of biotin. Absorbance 450 nm.

FIG. 24 provides sequence listings: SEQ ID. NO:1 amino acid sequence for IFN-γ scFV, SEQ ID. NO:2 nucleotide sequence for IFN-γ scFV, SEQ ID. NO:3 amino acid sequence for IFN-γ:CD19 Orientation Fusion Protein (Type A), SEQ ID. NO:4 nucleotide sequence for IFN-γ:CD19 Orientation Fusion Protein (Type A), SEQ ID. NO:5 amino acid sequence for CD19:IFN-γ Orientation Fusion Protein (Type B), SEQ ID. NO:6 nucleotide acid sequence for CD19:IFN-γ Orientation Fusion Protein (Type B), SEQ ID. NO:7 amino acid sequence for fusion protein tag sequence (Myc), SEQ ID. NO:8 nucleotide acid sequence for fusion protein tag sequence (Myc), SEQ ID. NO:9 amino acid sequence for fusion protein tag sequence (His), SEQ ID. NO:10 nucleotide acid sequence for fusion protein tag sequence (His), SEQ ID. NO:11 amino acid sequence for disulfide linker, SEQ ID. NO:12: nucleotide sequence for disulfide linker, SEQ ID. NO: 13 TNFascFV amino acid sequence, TNFascFV SEQ ID NO: 14 nucleotide sequence, SEQ ID. NO: 15 linker, SEQ ID. NO: 16: Linker, SEQ ID. NO: 17 TNF fusion protein and SEQ ID NO: 18 TNF fusion protein amino acid sequence.

FIG. 25 provides a schematic showing a representative bead-cell complex (including a fusion protein) (FIG. 25A), and a graph (FIG. 25B) showing that the least amount of CAR+ (as indicated by GFP expression) was present cells in the supernatant (after binding with beads, in the presence of IFN) when fusion protein is present (i.e. via the full complex shown in FIG. 25A).

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” refer to a relationship wherein structures are secured or attached to one another either directly or indirectly through intervening structures, as well as both movable or rigid attachments or relationships, unless expressly described otherwise.

For purposes of the description hereinafter, it is to be understood that the embodiments described below may assume alternative variations and embodiments. It is also to be understood that the specific articles, compositions, and/or processes described herein are exemplary and should not be considered as limiting.

In the present disclosure the singular forms “a,” “an,” and “the” include the plural reference, and reference to a particular numerical value includes at least that particular value, unless the context clearly indicates otherwise. Thus, for example, a reference to “a bead” or “a nano structure” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

As used herein, term “amino acid” broadly refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an l-amino acid.

As used herein, an “antibody fragment” includes a portion of an intact antibody, such as, for example, the antigen-binding or variable region of an antibody. Examples of antibody fragments include Fab, Fab′, F(ab′)₂, and Fv fragments. For example, antibody fragments include isolated fragments, “Fv” fragments (consisting of the variable regions of the heavy and light chains), recombinant single chain polypeptide molecules in which light and heavy chain variable regions are connected by a peptide linker (“scFv proteins”), recombinant single domain antibodies consisting of a variable region of an antibody heavy chain (e.g., VHH), and minimal recognition units consisting of the amino acid residues that mimic a hypervariable region (e.g., a hypervariable region of a heavy chain variable region (VH), a hypervariable region of a light chain variable region (VL), one or more CDR domains within the VH, and/or one or more CDR domains within the VL). In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, heavy chain variable region, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment.

As used herein, the term “binding” typically refers to a non-covalent association between or among two or more entities, unless expressed indicated otherwise, for example, referring to covalent bonding. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

The terms “Chimeric antigen receptor” or “CAR” or “CARs” as used herein refers to engineered receptors, which graft an antigen specificity onto cells (for example T cells such as naive T cells, central memory T cells, effector memory T cells or combination thereof). CARs are also known as artificial T-cell receptors, chimeric T-cell receptors or chimeric immunoreceptors. In some embodiments, CARs comprise an antigen-specific targeting regions, an extracellular domain, a transmembrane domain, one or more co-stimulatory domains, and an intracellular signaling domain.

The term “cytokine” as used herein refers to interferons (IFN), interferon gamma (IFN-γ), TNF-α interleukins, IL1 IL2 IL6, IL10, IL12, IL15, IL17, IL18, IL21 IL35, growth factors, CSF, chemokines, lymphokines and monokines

The term “domain” is used herein to refer to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively, or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecular (e.g., a small molecule, carbohydrate, a lipid, a nucleic acid, or a polypeptide). In some embodiments, a domain is a section of a polypeptide; in some such embodiments, a domain is characterized by a particular structural element (e.g., a particular amino acid sequence or sequence motif, α-helix character, j-sheet character, coiled-coil character, random coil character, etc.), and/or by a particular functional feature (e.g., binding activity, enzymatic activity, folding activity, signaling activity, etc.).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

As used herein, the term “fusion protein” generally refers to a polypeptide including at least two segments, each of which shows a high degree of amino acid identity to a peptide moiety that (1) occurs in nature, and/or (2) represents a functional domain of a polypeptide. Typically, a polypeptide containing at least two (or more) such segments is considered to be a fusion protein if the two segments are moieties that (1) are not included in nature in the same peptide, and/or (2) have not previously been linked to one another in a single polypeptide, and/or (3) have been linked to one another through action of the hand of man.

As used herein, the term “linker” refers to, e.g., in a fusion protein, an amino acid sequence of an appropriate length other than that appearing at a particular position in the natural protein and is generally designed to be flexible and/or to interpose a structure, such as an α-helix, between two protein moieties. In general, a linker allows two or more domains of a fusion protein to retain 50%, 55%, 60%, 65%, 70%, 75%, 80%, 85%, 90%, 95% or more of the biological activity of each of the domains. A linker may also be referred to as a spacer. The linker may comprise a flexible, rigid, enzymatically cleavable or in vivo cleavable disulfide linker. The linker may comprise a proline rich linker, (Gly4Ser)3 (SEQ ID NO:15), (GGGS) (SEQ ID NO:16) or LEAGCKNFFPRSFTSCGSLE (SEQ ID NO: 11). One or more linkers may be utilized depending on the intended purpose and nature of the fusion protein.

As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues.

As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

The abbreviations or acronyms used in the present disclosure have corresponding meanings as those skilled in the art understand. For example, the term “scFV” refers to a single chain antibody fragment. The terms “anti-IFN-γ scFV” and “IFN-γ scFV” may be used interchangeably, and the term “anti-” refers to an entity or portion targeting or to be bound with another entity or portion. The term “anti-IFN-γ scFV” refer to a single chain antibody fragment targeting or to be bound with IFN-7.

One approach for improving the clinical success of CAR-T cell therapies is to optimize the selection of specific CAR-T subpopulations and subsets. Such subpopulations and subsets include but are not limited to, CD4+ and CD8+ T cells, known as helper and cytotoxic T cells, respectively. Clinical studies have shown that CD4+ CAR-T cells collaborate with CD8+ CAR-T cells in tumor killing with the ratio of CD4+CD8+ cells being critical. Both CD4+ and CD8+ CAR-T cells express a polyfunctional set of Th1 and Th2 cytokines, such as IFN-γ and TNFα (TH1), IL-5, and IL-13 (TH2). Single-cell cytokine expression studies of CAR-T cells show that the levels of expressed cytokines such as IFN-γ are highly variable. Though not wishing to be bound by the following theory, it is though that expression of such cytokines by CAR-T cells can reflect an activated state, correlating with cell longevity and anti-tumor activity in patients.

Future development of CAR-T therapies must overcome variations in responses and unpredictable toxicity in patients, which can be partially attributed to interpatient heterogeneity of CAR-T products. The inventors herein have developed a novel approach for optimizing the selection of CAR-T products comprising the use of fusion proteins. In certain embodiments, the fusion proteins as described comprise one portion consisting of antibody fragments capable of binding to cytokines, fused to another portion consisting of a protein, peptide or fragment thereof, corresponding to a cancer antigen or tumor marker. The cytokines may include (but are not limited to) interferon gamma (IFN-γ) or tumor necrosis factor alpha (TNF-α). The cancer antigen may include, but is not limited to, CD19 extracellular domain (CD19-ECD).

In an embodiment, a fusion protein containing an IFN-γ scFv is fused to CD19-ECD and is used to assess CAR-T cells within a polyclonal population, based on their CAR expression levels. Characterization of such activity was carried out using SDS-PAGE for confirming size and glycosylation pattern, and ELISA assays for confirming specific binding to IFN-γ and FMC63 scFv. Binding of the fusion protein to anti-CD19 CAR expressing HEK293T and T cells was confirmed using flow cytometry and the sensitivity range was evaluated. Based on such studies (as further described in the Examples below) the novel fusion proteins of the invention can successfully identify individual CAR-T expressing cells. Furthermore, such fusion proteins may be used in cell therapy analytics (to determine the percentage of population that are high secreting) and as a process step, to isolate desired subpopulations with improved safety and efficacy.

In an embodiment, a fusion protein containing an TNF-α scFv is fused to CD19-ECD and is used to assess CAR-T cells within a polyclonal population, based on their CAR expression levels. Production and characterization of TNF-α.CD19 ECD fusion protein was performed with HEK293 cell transfection and ELISA variations (as further described in the Examples below). Results showed that the TNF-α scFv was capable of binding to TNF-α as demonstrated with a dose-dependent sigmoidal curve.

In an embodiment, cells other that HEK cells may be used to produce functional fusion proteins. Such cells may be selected on the basis of various criteria, including but not limited to, feasibility, scale-up ability and cost.

In an additional embodiment, the present disclosure, provides improved and cost-effective biomanufacturing of T cells for (autologous and/or allogeneic) immunotherapies enabling efficacious and uniform products. In certain embodiments, macro-sized beads for (scalable) packed beds that are surface functionalized to allow for isolation of the best or activated (CD4+) cells in a population (based on their ability to express/secrete the key cytokines IFNγ and TNF) may be utilized.

### 1. Compositions Structures of Fusion Proteins-IFN-γ:CD19

In certain embodiments, the extracellular portion of a CAR is derived from the variable light (V_{L}) and variable heavy (V_{H}) regions of an antibody (scFv) against the target of interest. The scFv affinity is a key design parameter for improving specificity of the CAR (on the surface of cancer cells) and for reducing off-target binding, and in the case of target antigens which are expressed on healthy tissue, reduce “on-target, off-tumor” side effects. For example, CARs which contained an anti-ErbB2 scFv with a K_{D} of 0.3 pM showed selective cytotoxicity towards highly expressing ErbB2 cells. The fusion proteins provided in the present disclosure can be adapted to bind to new CARs (and other immunotherapy cells) as they emerge.

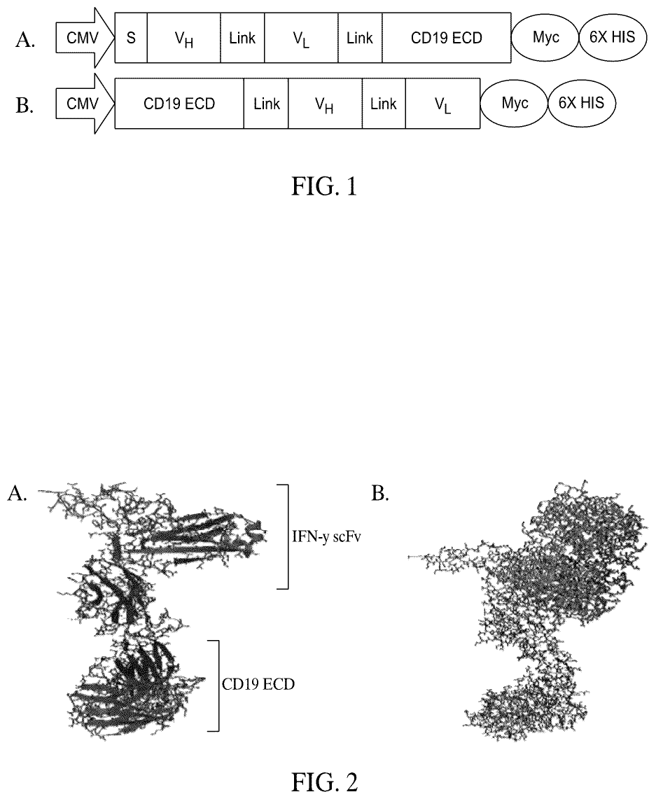

An exemplary novel protein of the invention is designated as an “IFN-γ:CD19 fusion protein.” This protein is designed to specifically capture secreted IFN-γ in CAR-expressing cells (hence may be referred to as “anti-IFN-γ scFV”). To accomplish this, the CD19 extracellular domain is fused with an extremely high affinity scFv specific for IFN-γ, with a (G₄S)₃ linker (SEQ ID NO:15). In accordance with certain embodiments, two fusion proteins, which are IFN-γ:CD19 fusion proteins, are evaluated, one with the CD19 on the N-terminal side, and a C-terminal IFN-γ scFv, the other in the reverse direction. (See amino acid sequences in the Sequence Listing ((SEQ ID NOS:1-6)). Both constructs contain a MYC tag (for fluorescent imaging) and a 6×His Tag (for purification by Nickel Chromatography) (FIG. 1).

As each domain of the fusion protein is essential for optimal function, careful consideration into the design of each domain is needed. First, in the fusion protein that contains an N-terminal IFN-γ scFv (FIG. 1A), there is a leader sequence (denoted S) upstream from the V_{H} region of the scFv. Such a leader sequence (denoted S) is not present in the reverse orientation construct. In this construct, the leader sequence is a CD8α signal peptide (MALPVTALLLPLALLLHAARP (SEQ ID NO: 11) for membrane targeting, enhancing the expression and secretion of the fusion protein. Next is the IFN-γ scFv which is derived from the therapeutic mAb, emapalumab (Gamifant, Novimmune SA). This mAb has an extremely high affinity (picomolar range) to both free and receptor bound IFN-γ, and is an inhibitor of IFN-γ function, so capturing IFN-γ will not subject the cells to extensive IFN-γ stimulation. Each of the V_{H} and V_{L} domains contain three complementarity-determining regions (CDRs) which generates their specificity towards IFN-γ and thus is where the scFv binds to their target. The full peptide sequences can be found in the Sequence Listing (SEQ ID NOS: 3 & 4).

The second functional domain of the fusion protein is the CD19 extracellular domain. Due to the necessity of binding the anti-CD19 CAR “Anchor” to the fusion protein, it is essential that the CAR scFv region can effectively bind to the chosen CD19 domain. The FMC63 scFv, which is utilized in the designed CAR (as described previously), has demonstrated high affinity binding towards the known human CD19 ECD sequence (K_{D}=0.32 nM). The human CD19 ECD sequence, is a 260 amino acid protein consisting of Pro20-Val279 region of CD19.

Another design element for the fusion protein comprises the linker (or linkers), which function to link the two functional domains together. An appropriate linker between the functional domains, comprises a linker that eliminates or minimizes misfolding of the protein, enables high protein production yield, and does not impair bioactivity. Linkers herein are selected according to such criteria and play an important role in the construction of a stable, bioactive fusion protein. There are three categories of linkers based on their structure: flexible, rigid, and in vivo cleavable linkers. In an embodiment, the fusion proteins of the invention comprise flexible linkers. Flexible linkers are required when joining domains which require movement or interaction and are composed of small amino acids. For example, one exemplary linker is the (GGGS)ₙ(SEQ ID NO:16) linker, where the “n” can be adjusted to achieve appropriate separation, and to allow for proper folding and biological activity. The (G₄S)₃ linker (SEQ ID NO:15) can be used to link the V_{H} and V_{L} regions of a scFv due to its high flexibility. Due to its simplicity, and flexibility the (G₄S)₃ linker (SEQ ID NO:15) is used to fuse the scFv to the ECD. In certain embodiments, longer or more rigid linker (such as proline rich linkers) instead if necessary (i.e. to improve fusion protein function).

Additionally, due to the fusion protein potential application as an isolation method, strategies to dissociate the fusion protein from the target cells are provided. One such method involves the use of in vivo cleavage linkers. One strategy is the reduction of disulfide bonds. One such disulfide linker, designed for recombinant fusion proteins (LEAGCKNFFPRSFTSCGSLE (SEQ ID NO: 11)), identified by Chen et al is based on a dithiocyclopeptide and has been shown to generate precisely constructed, homogenous products. One particular strategy is the use of in vivo cleavable linkers, which are designed to be cut by a specific protease via implementation of the protease-specific sequence into the linker. This strategy is used with antibody-drug conjugates, where the antibody is used to increase the stability of the drug.

To model the 3D structure of the fusion protein, homology modelling using SwissModel, in which an atomic-resolution model of the fusion protein is generated from comparing the amino acid sequence to a related homologous protein template. Although there are limitations with this technique, due to the reliance on high sequence alignment for accurate structural modeling, it can be reasonably assumed that the structure resembles that shown in FIG. 2. Although the interaction between IFN-γ and the fusion protein scFv was not generated using a model, the basic interaction demonstrating the relative location of the binding site, and the comparative size of the fusion protein is achieved.

### 2. Steps for Making Fusion Protein IFN-γ:CD19 ECD

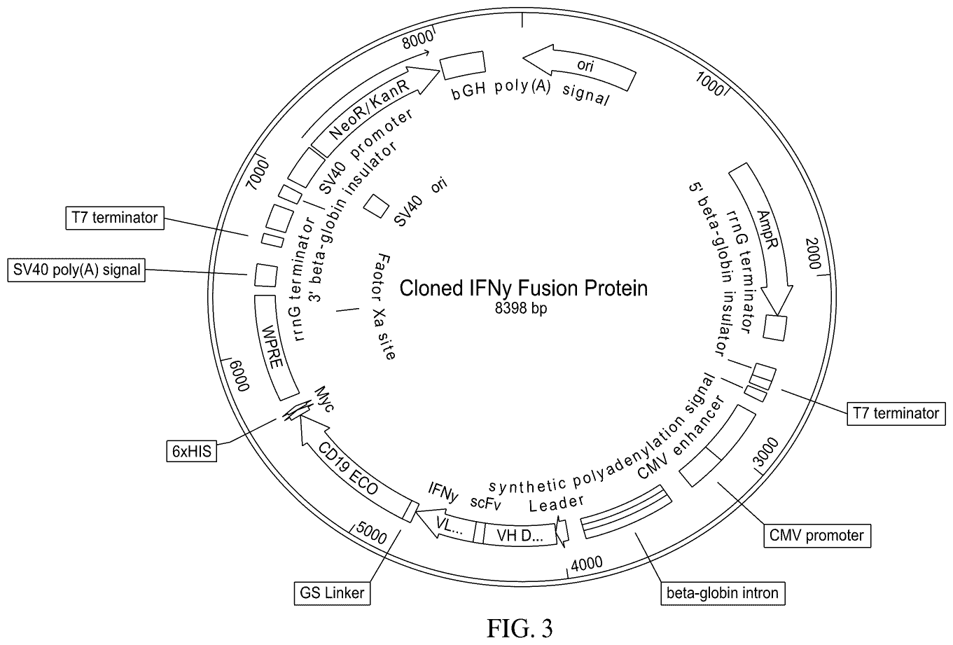

The fusion proteins described herein are produced via transient expression in human embryonic kidney (HEK) 293T cells using an expression vector (as shown in FIG. 3) with a CMV promoter. Mammalian cells, HEK 293T and Chinese Hamster Ovary (CHO) cells were evaluated for use as expression systems for production of recombinant proteins due to their ability to generate human glycosylation patterns. CHO cells are challenging to transiently transfect, and although are mammalian cells, they are still non-human, and as such are often not use for research purposes. (CHO cells remain the gold standard for large scale manufacturing of recombinant proteins and may be utilized in further evaluation studies.) HEK293 cells are the human cell line most often used in the production of biotherapeutic proteins and offer a fully human glycosylation pattern, thus reducing potential toxicity. HEK293 cells are highly amenable to transient transfection, and due to their ease of use are the most common expression system for research-grade protein production. FDA approved recombinant fusion proteins produced in HEK 293 cells, provides further support for the choice of HEK293 cells as expression system for the fusion proteins of the current disclosure.

In accordance with standard protocols HEK293T cells were seeded twenty-four hours before transfection to achieve ˜80% confluency at the time of transfection. On the day of transfection, 1.5 μL Lipofectamine 3000 transfection reagent (ThermoFisher) was diluted in 25 μL Opti-MEM Reduced Serum Medium and vortexed briefly. A master mix was prepared by diluting 500 ng of pTwist-Fusion in 25 μL Opti-MEM Reduced Serum Medium, and 1 μL of P3000 Reagent and vortexed briefly. The diluted pTwist-Fusion DNA was added to the diluted Lipofectamine 3000 Reagent (1:1 Ratio) and incubated for 10-15 minutes at room temperature. Following incubation, 50 μL of the DNA-Lipid complex was added to the HEK293T cells and incubated for 2-4 days at 37° C. and 5% CO₂. It should be noted that reagent volumes and DNA quantity mentioned was on a per well basis for a 24-well plate and was scaled according to experimental conditions.

Following incubation, IFN-γ:CD19 ECD fusion protein purification was performed using a Ni-NTA Spin Column (Qiagen) under native conditions according to manufacturer's protocol. Briefly, HEK293T culture supernatant was collected and loaded onto a pre-equilibrated Ni-NTA spin column and centrifuged to bind 6×His-tagged protein to resin. Ni-NTA spin column was equilibrated using buffer containing 10 mM imidazole. Following binding, column was washed twice with buffer containing 20 mM imidazole to ensure removal of contaminants and the flow through was saved for SDS-PAGE analysis. Fusion protein was eluted by washing twice with a buffer containing 500 mM imidazole and protein concentration was determined using spectrophotometer (A₂₈₀) and Bradford Assay.

### 3. Steps for Using Fusion Protein to Isolate Selected T Cells

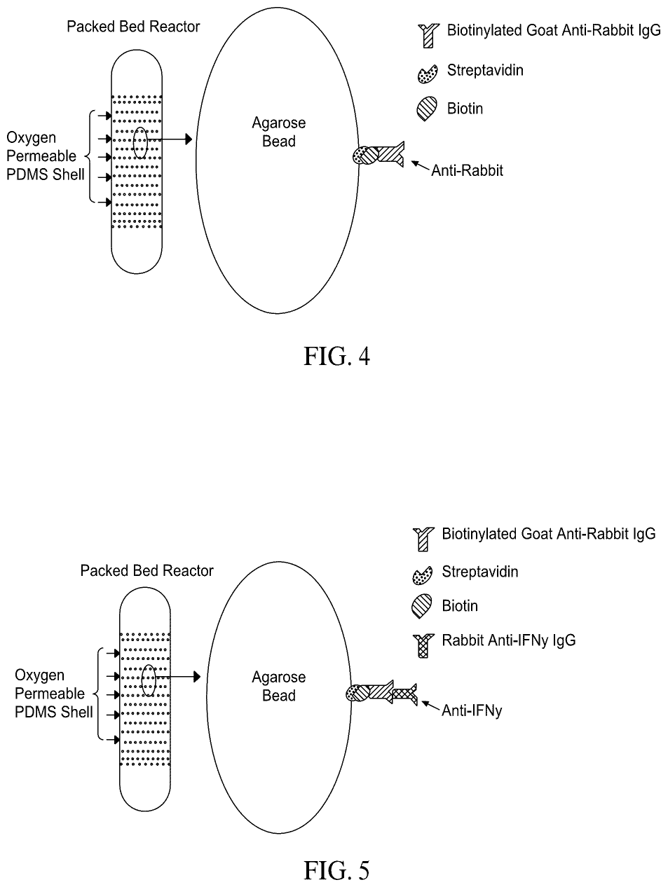

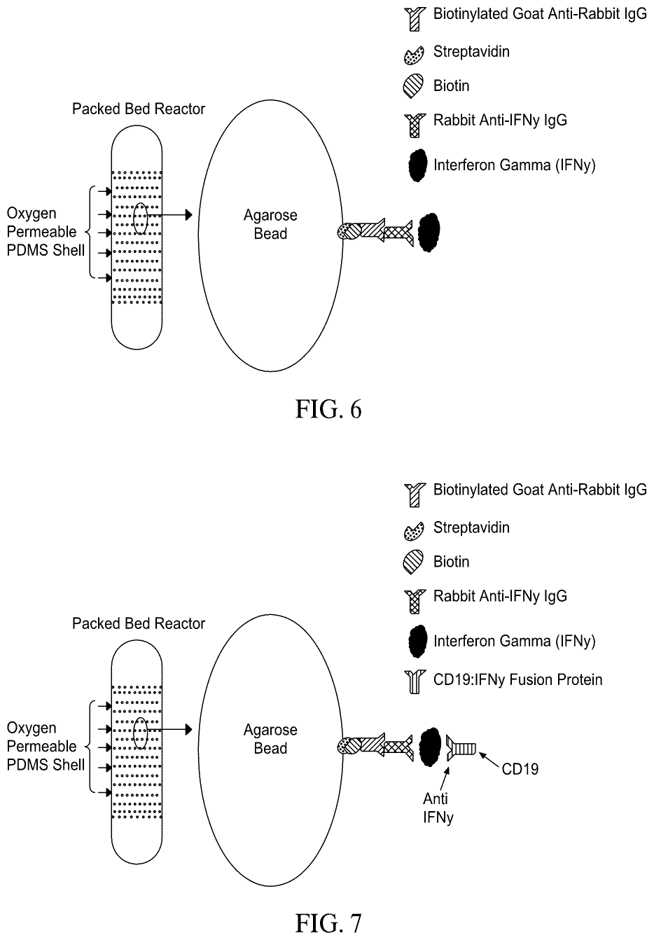

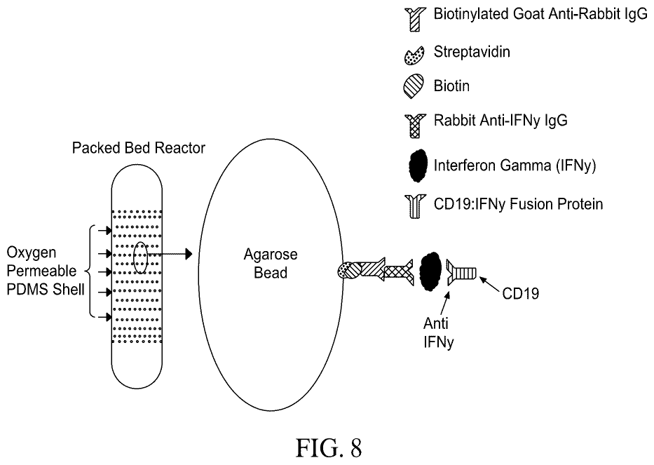

In FIGS. 4-8, like items are indicated by like reference numerals, and for brevity, descriptions of the structure, provided above with reference to the preceding figures, are not repeated. The method of using the fusion protein and the method for isolating or purifying CAR-T-cells are described with reference to the exemplary structure described in FIGS. 4-8.

Two FDA approved anti-CD19 therapies, Yescarta© and Kymriah© are constructed using the murine-derived FMC63 anti-CD19 scFv, which has a relatively high affinity—K_{D} of 0.32 nM. As described herein, IFN-γ scFv: CD19 fusion protein is evaluated first, and added to a mix of human T cells being expanded as per a typical CAR-T process. One side of the fusion protein binds to the CAR receptor on the external surface of a given T cell, and then the other side of the fusion protein binds to the IFN-γ being secreted by that same T cell. The bound IFN-γ on the T cell surface then binds to an antibody, which can be attached to the surface of a bead/particle, allowing for isolation of these high-secreting T cells from the T cells in the population producing lower amounts of IFN-γ. This approach allows for production of a CAR-T product that is enriched in activated T cells that produce optimal levels of important cytokines (i.e. IFN-γ etc.).

In accordance with certain embodiments, it is proposed to develop novel macro-sized (0.3-3 mm) beads for use in packed bed bioreactors and columns to selectively isolate IFN-γ high producing CAR-T-cells. With beads of this (macro) size, T cells (6-7 um in diameter) are able to flow through beds (at any process scale) packed with such beads (see FIG. 4) and not plug the column. In FIGS. 4-8, “agarose bead” (or beads) are shown for the purpose of illustration only. Such beads can be made any suitable polymers, which can be cross-linked and porous. For example, the polymers may be made of repeating units of agarobiose or styrene in some embodiments. Previous research has demonstrated that 3 mm diameter by 2.5 mm length fused polystyrene cylindrical pellets are of sufficient size for expanding mesenchymal stem cells without leading to packed bed blockage, which led to the choice in size for these beads.

In designing the immobilization support, the inventors purposefully selected, tested and engineered beads and matrices having optimal utility for use with fusion proteins for ultimately enabling CAR T selection. In protein coupling, specifically antibody coupling, suitable examples include (but are not limited to) agarose and polystyrene. Agarose is a natural polysaccharide matrix which has a primary structure consisting of alternating D-galactose and 3-anhydrogalactose. The large accessible pore structure supplies an increased capacity for affinity immobilization. When coupling antibodies to an agarose support, the agarose is first activated via cyanogen bromide (CNBr) method which produces cyanate esters on the agarose surface, thus providing an effective surface for coupling to primary amine groups. These activated agarose beads can then be coated with streptavidin, allowing the use of the high affinity avidin-biotin interaction for binding and elution. The initial bead experiments utilize commercially available agarose beads that are coated with avidin. The streptavidin can be covalently coupled to the bead surface via two step 1-ethyl-3(-3-dimethylaminopropyl) carbodiimide hydrochloride (EDC) coupling as proposed in Particle Coating Procedures (Spherotech, Inc).

Biotinylated goat anti-rabbit IgG secondary antibody is immobilized on the macro beads through interaction with the streptavidin coated particles. FIGS. 4-8 outline the proposed mechanism, by which the IFN-γ high expressing CD8+ T cells are isolated using the novel affinity chromatography approach. The IFN-γ:CD19 fusion protein binds to both the T cell surface bound anti-CD19 CAR, and secreted IFN-γ. A rabbit polyclonal anti-IFN-γ antibody binds to captured IFN-γ, stabilizing the immune complex. A column is packed with macro beads coated with biotinylated goat anti-rabbit IgG, which binds to the rabbit polyclonal mAb, and thus captures the cells expressing IFN-γ. The CAR-T cell suspension (following expansion) is pumped onto the column and flowed through the column via gravity flow, or by pumping at low pressure. Thus, the highest producing CAR-T cells are captured by the column, whereas cells lacking in production are not captured by the column and flow through.

When conditions have been adjusted to adequately identify IFN-γ high expressing CAR-T cells, resin binding experiments are performed. Cell incubation conditions are established for capture. A variety of approaches can be used or evaluated for release of bound complexes, including: a.) outcompeting with excess biotin; b.) use of in vivo cleavable linkers which are designed to be cut by a specific protease via implementation of the protease-specific sequence into the linker. This strategy is used with antibody-drug conjugates, where the antibody is used to increase the stability of the drug. For option a.) washing with a concentrated biotin elution buffer leads to disruption of the biotinylated goat anti-rabbit IgG-Strep binding interaction, thus releasing cells from the column. The anti-goat IgG bound to the cells subsequently dissociates due to their low affinity.

As described herein, the processes for manufacturing and using the novel fusion proteins of the invention for selecting homologous populations of T cells, such as CAR T cells, are suitable and appropriate for large scale manufacturing. Unlike the common current approach offered for isolation of T cells by manufacturers like Miltenyi which use magnetic beads for small batches in autologous cell therapy processes (that require large expensive magnets, both unusual and unconventional, for use in a large scale biopharmaceutical production process) the methodology employed herein comprising the use of column bind/elute chromatography is both accessible and practical for routine use.

(4) Measuring Secreted Proteins at Single-Cell Level with On-Cell Methods (OCMS)

The OCMS method is used for capturing and screening monoclonal antibodies (mAbs) on the surface of hybridoma cells to find rare, high-value human mAbs. OCMS hybridomas express an anchor protein on their cell surface. The cells are incubated with a linker molecule, which attaches to the anchor. The linker is bi-functional; it binds to both the anchor and the secreted protein of interest. Human IgG mAb's captured on the cell surface can be tested for antigen binding by incubating the hybridomas with fluorescent antigens, which thus labels the cells making the protein of interest. The reaction is specific because there is an excess of linker in the reaction—any mAbs that escape the cell that made them are saturated with the linker and thus prevented from binding to any other cells.

For this invention, the membrane bound anti-CD19 CAR serves as the anchor and the proposed fusion protein as the linker, thus the fusion protein's CD19 ECD binds to the CAR, and the IFN-γ scFv binds secreted IFN-γ. Excess fusion protein in the cell culture medium acts as a competitor blocking the binding of secreted IFN-γ to cells which do not secrete. This technology is utilized to identify cells that secrete cytokines.

As detailed in the Examples, the inventors have herein discovered and invented novel fusion proteins that can be utilized in methods to recognize and capture CAR-T cells based on CAR expression levels. The invention enables much needed methods for making a CAR product of uniform composition and efficacy by use of novel fusion proteins to select CAR-T cells with high levels of targeted expression and thereby overcomes challenges associated with viral delivery methods. In an additional aspect, the fusion proteins of the invention are particularly useful as not only are they engineered to detect CARs with a high level of specificity, they are also engineered to detect (and select) CARs that have certain cytokine expression. In an embodiment, the novel fusion proteins of the invention are designed to recognize CARs that express IFN-γ. The fusion proteins uniquely recognize the CARs and cytokine expression levels in tandem. As described herein, this fusion proteins of the invention have the potential for use in cell therapy analytics (to determine the percentage of population that are high secreting) and as a process step, to isolate desired subpopulations with improved safety and efficacy.

### 5. Compositions Structures of Fusion Proteins-TNF-α:CD19

TNF-α is an inflammatory cytokine capable of stimulating effector T cell differentiation, proliferation, and induce cell death. It has shown a strong correlation for enhancing TCR-mediated activation of both CD4+ and CD8+ T cells. It can also affect the suppressive activities of regulatory T cells in addition to their development through TNFR2 receptors in CD4+ cells. This is why medications that suppress TNF-α expression can have dramatic effects on the relationship between the effector and regulatory T cells, which affects the immune system as a whole. When released, TNF-α can be in soluble and membrane-bound form. Engineered CAR T cells are no different, where cytokines TNF-α and IFN-γ are released upon ligation of the binding domain with the antigen of interest, in addition to granzyme and perforin to perform cytolysis.

As discussed above, previous attempts isolating cells in polyclonal population have included Secretion Capture Report Web (SCRW), Gel Microdroplets, and Semi-Solid Medium. Though somewhat effective, the secondary mAbs in such methods that identify secreting hybridomas encapsulated in agarose drops occasionally bind to nonsecreting cells. These challenges are overcome with the novel fusion proteins of the present disclosure.

As described in detail in the Examples, production and characterization of TNF-α:CD19 ECD fusion protein was performed with HEK293 cell transfection and ELISA variations. Results showed that the TNF-α scFv was capable of binding to TNF-α as demonstrated with a dose-dependent sigmoidal curve. Further, the TNF-α scFv outcompeted an anti-TNF-α mAb that was allowed to bind to TNF-α before fusion protein introduction, where only a 53% difference in absorbance was seen comparing wells with and without competition. A decrease was expected because antibodies have better binding capabilities with two scFv's and pose steric hinderance with a larger 150 kDa, compared to 62 kDa molecular weight of fusion protein. Absorbance in competitive wells indicated fusion protein binding regardless, demonstrating strong binding adequate for fusion protein performance when incubated with live CAR T cells. Finally, an FMC63 ELISA demonstrated fusion protein was captured by FMC63, indicating that the CD19 ECD is functional and available for anti-CD19 scFv capture. With both key binding domains confirmed functional, characterization was demonstrated to be successful. Accordingly, the novel TNF-α scFv fusion protein described herein is an additional example of means by which to accurately identify and later isolate individual CAR T cells displaying high efficacy, as determined by release of soluble cytokines, in this case, TNF-α, upon ligation of the binding domain to cancer antigen CD19. This molecule is a TNF-α: CD19 fusion protein that consists of a TNF-α scFv and CD19 extracellular domain linked with (Gly₄Ser)₃(SEQ ID NO:15) peptide linker.

In an embodiment, provided herein are novel fusion proteins comprising a first portion consisting of at least one single chain antibody fragment (scFV), and a second portion consisting of an antigen, such as a cancer antigen or marker, or a fragment thereof. The scFV is capable of selectively binding to a cytokine released by chimeric antigen receptor (CAR) expressing cell. The cancer antigen or marker may comprise an extracellular domain of CD19. The fusion protein may further comprise at least one or more linkers between scFV and the extracellular domain of CD19.

In certain embodiments, the scFV selectively binds to cytokines such as (but not limited to) interferons, interferon gamma (IFN-γ), TNF-α,interleukins, IL1 IL2 IL6, IL10, IL12, IL15, IL17, IL18, IL21 IL35, growth factors, CSF, chemokines, lymphokines and monokines. The cytokines may consist of interferon gamma (IFN-γ) or TNF-α.

In an embodiment, the cytokine released by chimeric antigen receptor (CAR) expressing cell consists of interferon gamma (IFN-γ), and is recognized by the scFV comprising the amino acid sequence of SEQ ID NO:1. A fusion protein comprising the amino acid sequences of SEQ ID NO: 3 or SEQ ID NO:5 may be used to selectively bind a chimeric antigen receptor (CAR) expressing cell that releases interferon gamma (IFN-γ).

The fusion proteins of the disclosure may further comprise a flexible linker, a rigid linker, an enzymatically cleavable linker, an in vivo cleavable disulfide linker or a linker comprising protease-specific sequences. In certain embodiments, the linker comprises a proline rich linker, (Gly4Ser)3(SEQ ID NO:15), (G₄S)₃(SEQ ID NO:15), (GGGS) (SEQ ID NO:16) or LEAGCKNFFPRSFTSCGSLE (SEQ ID NO: 11)).

In an embodiment, the cytokine released by chimeric antigen receptor (CAR) expressing cell consists of tumor necrosis factor alpha (TNF-α), and is recognized by the scFV comprising the amino acid sequence of SEQ ID NO: 13, and nucleotide sequence of SEQ ID NO: 14. A fusion protein comprising the amino acid sequences of SEQ ID NO: 13 may be used to selectively bind a chimeric antigen receptor (CAR) expressing cell that releases TNF-α.

In an embodiment, the novel fusion proteins of the invention may be used for isolating or purifying a CAR expressing cell, wherein the scFV of the fusion protein binds with a cytokine secreted from a chimeric antigen receptor (CAR) expressing cell, and the extracellular domain of CD19 binds with an anti-CD19 chimeric antigen receptor (CAR). The cytokines secreted/released by such chimeric antigen receptor (CAR) expressing cells may comprise interferons, interferon gamma (IFN-γ), TNF-α,interleukins, IL1 IL2 IL6, IL10, IL12, I15, IL17, IL18, IL21 IL35, growth factors, CSF, chemokines, lymphokines or monokines.

In an embodiment, the isolation or purification of a CAR expressing cell comprising the use of the novel fusion proteins described herein, is performed in a bed reaction packed with beads made of a polymer.

In an embodiment, the novel fusion proteins of the invention, comprising at least single chain antibody fragment (scFV), and a cancer antigen or marker, or a fragment thereof, may be used to selectively identify immunotherapy cells of interest, such as those releasing specific cytokines such as IFN-γ or TNF-α. In an embodiment, the novel fusion proteins may be utilized for selecting specific CAR T cells from polyclonal samples enabling identification and selection of immunotherapy cells having particular characteristics. The novel fusion proteins described herein further enable the characterization of immunotherapy cells, such as those that secrete specific cytokines, and also enable the subsequent identification and isolation of specific cytokine secreting cell populations.

In an embodiment, the fusion proteins of the disclosure enable the selection of defined subsets of T cells prior to transduction and further allow for the production of therapeutic CAR-T products of uniform composition, thereby enabling reproducible potency, aiding in determining potential correlations in cell dose and efficacy and toxicity during clinical trials.

The following examples are given to illustrate exemplary embodiments of the present disclosure. It should be understood, however, that the present disclosure is not to be limited to the specific conditions or details described in these examples. Examples are provided below to facilitate a more complete understanding of the invention. The following examples illustrate the exemplary modes of making and practicing the invention.

### EXAMPLES

### Example 1

### Construction and Evaluation of IFN-γ & CD19 Fusion Proteins

### Materials and Methods

(1) Construction of Fusion Protein Production Plasmids: The fusion protein gene construct, which contained an IFN-γ scFv and CD19 ECD linked via a flexible peptide (Gly4Ser)3(SEQ ID NO:15) linker, along with a Myc- and 6×-His Tag was cloned into pTwist-CMV-BetaGLobin-WPRE-Neo (Twist Bioscience) using EcoRI and XhaI restriction sites by Twist Bioscience (San Francisco, CA, USA). The resulting vector was named pTwist-Fusion. Two fusion proteins were evaluated, one with the IFN-γ scFv on the N-terminal side, and a C-terminal CD19 ECD, the other in the reverse direction (Type A and Type B).

The Type A and B sequences are slightly different in that they have different orientations. The fusion protein includes both the IFN-γ scFv (Vh-Link-Vl) linked to the CD19ECD. The Myc and 6×his tags are part of the fusion protein; however, they are there for isolation purposes during protein production and not relevant for the function of the protein (i.e., different domains could be used instead) and so not included in the sequence(s). The sequences listed as SEQ ID NOS: 3, 4, 5 and 6 include: both the heavy and light chains for the IFN-γ scFv, the first G4S3 (SEQ ID NO:15) linker between the two variable regions, but not the linker between the two protein domains and the CD19 ECD. (The sequence for the ECD is a human CD19 ECD sequence—a 260 amino acid protein consisting of Pro20-Val279 region of CD19 (UniProt accession #P15391)).

(2) Cell Culture: HEK 293T cells (ATCC No. CRL-3216) were cultured in high-glucose containing Dul-becco's modified Eagle's medium (DMEM; Lonza) supplemented with 10% fetal bovine serum (FBS; Gibco), 50 U/mL penicillin, and 50 pg/mL streptomycin. Jurkat cells (ATCC TIB-152) were cultured in RPMI medium (ATCC) containing 10% FBS (Gibco), 50 U/mL penicillin, and 50 pg/mL streptomycin. CAR-T cells were generously provided by the University of Pennsylvania and were cultured in XVIVO-15 medium (Lonza) kept at a constant 5×10⁵ cells/mL with the medium replaced every three days.

(3) Transient Transfection: For transient expression of the fusion proteins, 1.5×10⁵ HEK 293T cells were transfected using the standard manufacturer's Lipofectamine 3000 protocol with 0.5 pg of the pTwist-Fusion plasmid in 24-well plates containing 1 mL complete growth medium and incubated for 48 hours. After 48 hours, culture medium was harvested, and cell culture was replenished with 1 mL fresh complete growth medium and incubated for another 24 hours. After 24 hours, the culture medium was harvested again. Harvested protein samples were purified using Ni-NTA purification kit (Qiagen) according to manufacturer's protocol. For transient expression of the FMC63-Fc protein, 1.5×10⁵ HEK 293T cells were transfected using the standard manufacturer's Lipofectamine 3000 protocol with 0.5 pg of pcDNA3.1(-) FMC-63 scFv-Fc (huFc) (AddGene Plasmid #183252) following the same procedure described above.

(4) SDS-PAGE Gel Electrophoresis: To evaluate glycosylation of the fusion protein, a reduced, denaturing protein electrophoresis was performed running 1-10 pg of the purified fusion protein sample following treatment with Protein Deglycosylation Mix II (New England Biolabs Inc, Ipswich, MA); reducing conditions were performed mixing each purified sample with 6.25 μL Nu-PAGE™ Sample Reducing Buffer (Invitrogen, Carlsbad, CA), 2.5 μL NuPAGE™ Sample Reducing Agent (Invitrogen, Carlsbad, CA), and heating at 70° C. for 10 minutes before electrophoresis on NuPAGE™ 4-12% Bis-Tris Mini Gels 1.0 mm (Invitrogen, Carlsbad, CA). The bands were visualized by Coomassie blue staining. All procedures were performed according to the manufacturer's instructions.

(5) Biotinylation of the Fusion Protein: Purified fusion protein was biotinylated using the EZ-Link Sulfo-NHS-LC-Biotinylation Kit (ThermoFisher) according to manufacturer's protocol. Quantification of biotinylation was performed using the Pierce Biotin Quantification Kit (ThermoFisher) according to manufacturer's protocol.

(6) Enzyme-Linked Immunosorbent Assay: For initial recombinant IFN-γ binding, flat-bottomed 96-well MaxiSorp plates (NUNC, Rochester, NY) were coated with 500 ng of either orientation of fusion protein in 100 μL PBS and incubated overnight in 4° C. The plates were washed 3× with 200 μL PBS+0.05% Tween-20 (PBST) and then blocked in 100 μL PBST+5% dry non-fat milk powder for 1 hr at 37° C. After three washes with PBST, the plates were incubated for 1 hr with various concentrations of recombinant IFN-γ (StemCell, Seattle, WA) diluted in 100 μL PBS at 37° C. Following three washes with PBST, 500 ng of biotinylated mouse anti-human IFN-γ mAb (SouthernBiotech, Birmingham, AL) diluted in 100 μL PBS and incubated for 1 hr at 37° C. Following three washes with PBST, plates were incubated for 1 hr at 37° C. with 100 μL of a 1:1000 dilution of streptavidin conjugated to horseradish peroxidase (SA-HRP; BioLegend, San Diego, CA). Following three washes with PBST, plates were developed for 15 min at room temperature in the dark with 100 μL of TMB substrate (ThermoFisher) and stopped with equal volume of stop solution (ThermoFisher). The stopped reactions were assayed by spectrophotometry at 450 nm using an Epoch2 microplate reader (Agilent, Santa Clara, CA).

For the competitive binding evaluation, flat-bottomed 96-well MaxiSorp plates (NUNC, Rochester, NY) were coated with 500 ng of IFN-γ (StemCell, Seattle, WA) diluted in 100 uL PBS and incubated overnight in 4° C. The plates were washed 3× with 200 μL PBS+0.05% Tween-20 (PBST) and then blocked in 100 μL PBST+5% dry non-fat milk powder for 1 hr at 37° C. After three washes with PBST, the plates were incubated either with 500 ng of rabbit anti-human IFN-γ polyclonal mAb (Bio-Rad, Hercules, CA) diluted in 100 μL PBS, or 100 μL PBS for 1 hr at 37° C. Following three washes with PBST, entire plate was coated with 500 ng of biotinylated fusion protein diluted in 100 μL PBS and incubated for 1 hr at 37° C. Addition of SA-HRP and substrate followed procedure outlined above.

To evaluate FMC63 scFv binding, flat-bottomed 96-well MaxiSorp plates (NUNC, Rochester, NY) were coated with 500 ng of FMC63-Fc diluted in 100 μL PBS and incubated overnight in 4° C. The plates were washed 3× with 200 μL PBS+0.05% Tween-20 (PBST) and then blocked in 100 μL PBST+5% dry non-fat milk powder for 1 hr at 37° C. After washing with PBST, the plates were incubated with 500 ng of either orientation of biotinylated fusion protein diluted in 100 μL PBS for 1 hr at 37° C., with 100 μL PBS as a negative control. Following three washes with PBST, the addition of SA-HRP and substrate followed procedure outlined above.

(7) Flow Cytometry: For in vitro fusion protein binding evaluation, round-bottom 96-well plates were blocked with 200 μL PBS+1% BSA blocking buffer at room temperature for 1 hour. Cells were fixed with 4% paraformaldehyde for 20 minutes at room temperature. Following blocking and fixing, 1×106 cells were plated and incubated in 100 μL PBS+1% BSA for 30 minutes at room temperature. Various concentrations of biotinylated fusion protein were added to cells and incubated for 1 hour on ice in 50 μL staining buffer (BioLegend). After 3× wash with blocking buffer, 1:10-1:100 PerCp-streptavidin (BioLegend) was added and incubated for 20-30 minutes on ice. Following 3× wash with staining buffer, analysis was performed using the Guava easyCyte™ flow cytometer (Millipore Sigma; Burlington, Massachusetts) and post-processing was performed using InCyte™ software. K-means clustering was performed using Floreada.io software.

### Results

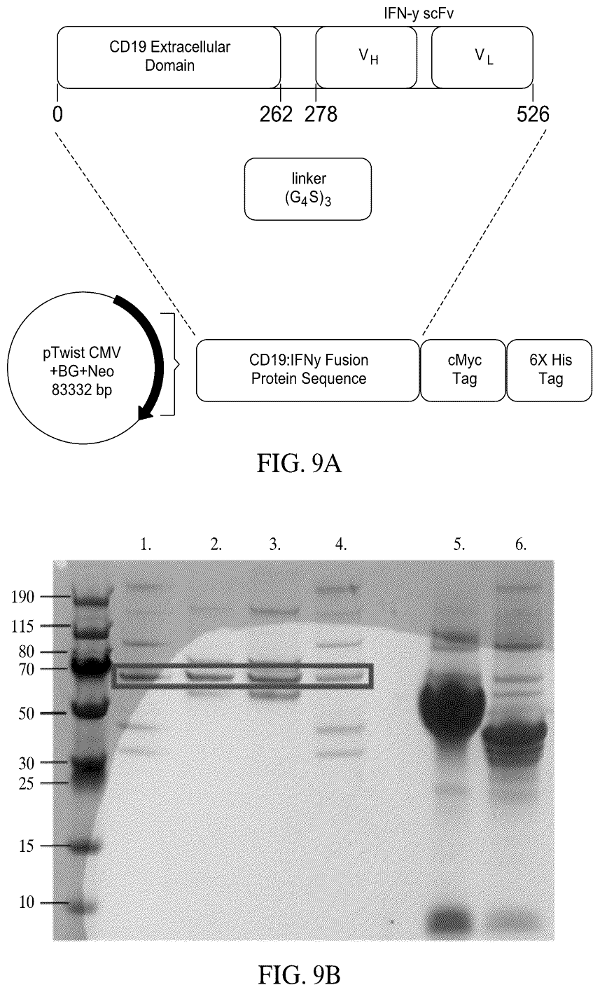

(1) Construction of IFN-γ scFv:CD19 ECD and CD19 ECD: IFN-γ scFv Fusion Protein Expressing Plasmids: As discussed previously, the fusion protein contains two key domains. First, an IFN-γ scFv which was derived from the therapeutic mAb, Emapalumab [25]. The extracellular domain for human CD19 protein is coded by exons 1-4 and contains two immunoglobulin-like domains [26-28]. The two fusion protein orientation fragments, with either the IFN-γ scFv on the N-terminal or C-terminal, were cloned into the pTwist-CMV-BG-WPRE plasmid, driven by the CMV promoter (FIG. 9A).

(2) SDS-PAGE Gel Electrophoresis: From calculations based on the amino acid sequences, the fusion proteins IFN-γ scFv:CD19 ECD and CD19 ECD: IFN-γ scFv should have molecular masses of 59 kDa and 58 kDa respectively. FIG. 9B shows a reduced, denaturing SDS-PAGE electrophoresis gel with the two fusion protein orientations isolated from producer cell supernatants, confirming expected findings. However, in addition to the expected monomer-sized band for each fusion protein observed, a band slightly larger and a band slightly smaller were present which likely represents glycosylated forms. To evaluate whether the larger bands were due to glycosylation, enzymatic deglycosylation was performed and the resulting SDS-PAGE gel (FIG. 9B) confirms the presence of N-linked glycosylation of the native fusion protein. The degylcosylated fusion protein lanes included the expected monomer-sized band, as well as bands which represent the deglycosylation enzymes.

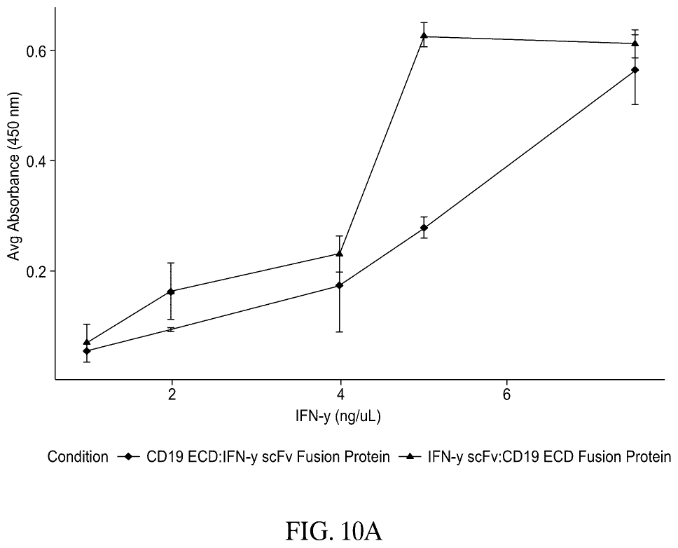

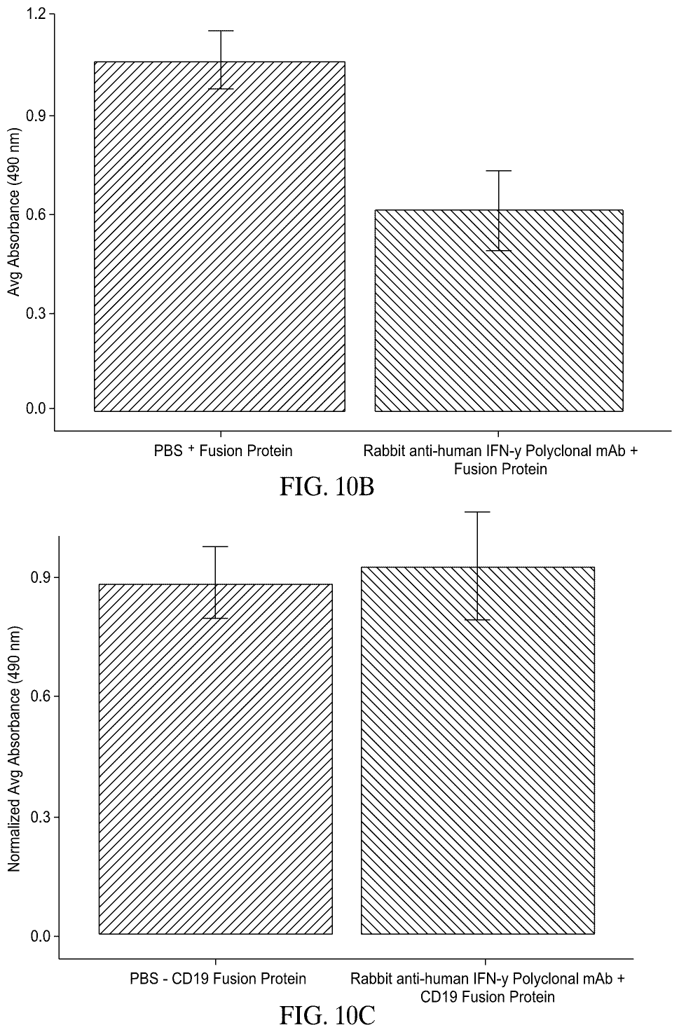

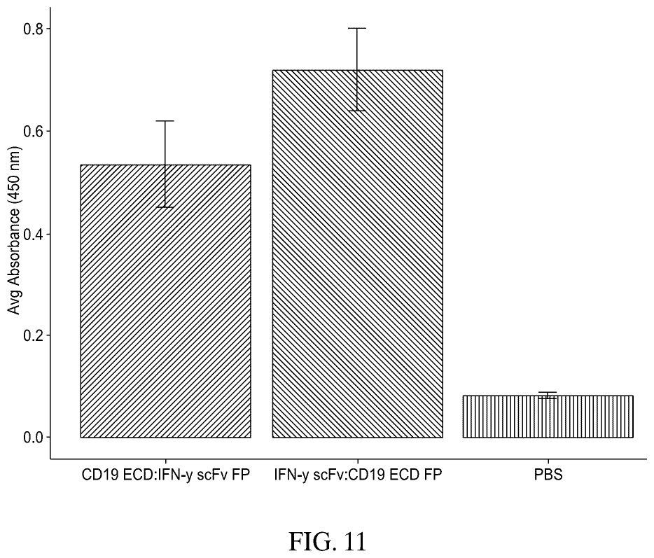

(3) Recombinant IFN-γ Binding: Sandwich ELISA assays were used to assess the presence, and binding affinity of the IFN-γ scFv domain on both fusion protein orientations. The specific binding of the fusion protein to soluble IFN-γ was evaluated using biotinylated fusion proteins as the capture antibody with a range of IFN-γ (FIG. 10A). Both fusion protein orientations bound to soluble IFN-γ in a dose-dependent fashion, with both orientations having a similar binding profile. The exception however was with 5 ng/μL IFN-γ where the IFN-γ scFv: CD19 ECD fusion protein had substantially higher binding (A450 nm=0.628±0.021) than the CD19 ECD: IFN-γ scFv fusion protein (A450 nm=0.279±0.018). Despite this difference, these results present evidence that the IFN-γ scFv is biologically active in both orientations of fusion proteins and capture of soluble IFN-γ can be achieved using the novel fusion proteins.

To further investigate IFN-γ binding, a competitive binding assay was performed using 5 ng/μL rabbit anti-human IFN-γ polyclonal mAb as a competitor to 5 ng/μL biotinylated IFN-γ scFv: CD19 ECD fusion protein binding 500 ng IFN-γ (FIG. 10B). There was a statistically significant difference in binding of the fusion protein in the presence of the competitor (M=0.612, SD=0.1211) compared to without the competitor (M=1.065, SD=0.088); t(3.67)=5.23, p=0.008. This decrease in net absorbance corresponded to a 43% decrease in binding with the competitor. Although it is expected that the fusion protein binding would be reduced in the presence of the competitor, due to the nature of the competitor being dimeric compared to the monomeric fusion protein, these results indicate that the binding affinity of the fusion protein is similar to that of the competitor.

Similarly, a competitive binding assay was performed on the reverse orientation fusion protein CD19 ECD: IFN-γ scFv (FIG. 10C). There was not a statistically significant difference in binding in the presence of the competitor (M=0.925, SD=0.136) compared to without the competitor (M=0.884, SD=0.089), t (3.45)=−0.438, p=0.687. These results indicate that the CD19 ECD: IFN-γ scFv fusion protein showed unnoticeable binding in the presence of the inhibitor. Despite the similar overall binding properties of these two fusion protein orientations, previous results showed drastic differences in the binding with 5 ng/μL of fusion protein, so these results are not surprising.

(4) FMC63 Binding: Most anti-CD19 CARs utilize an anti-CD19 scFv that was derived from the FMC63 mouse hybridoma, thus it is critical that the CD19 ECD in the fusion protein is functional, can be recognized and bound by FMC63. To evaluate the CD19 ECD functionality, recombinant FMC63-Fc protein was produced and used as the capture antibody in a direct ELISA (FIG. 3). Both fusion protein orientations were captured by the FMC63-Fc indicating that the CD19 ECD in the fusion protein is functional and can be recognized by FMC63. A Welch two-sample t-test was performed to compare the binding (via absorbance) between CD19 ECD: IFN-γ scFv or IFN-γ scFv: CD19 ECD with the negative control (PBS). There was a statistically significant difference in binding between the CD19 ECD: IFN-γ scFv (M=0.537, SD=0.084) and PBS only (M=0.0833, SD=0.005); t(2.0181)=9.294, p=0.011. Additionally, there was a statistically significant binding between the IFN-γ scFv: CD19 ECD fusion protein (M=0.721, SD=0.080) and PBS alone (M=0.0833, SD=0.005); t(2.02)=13.73, p=0.005. There was not a significant difference in binding between CD19 ECD: IFN-γ scFv (M=0.537, SD=0.084) and IFN-γ scFv: CD19 ECD (M=0.721, SD=0.080); t (3.99)=-2.7399, p=0.05205. These results indicate that the CD19 ECD on both orientations is functionally active, with the binding sites available for binding to the FMC63 scFv.

(5) In vitro Fusion Protein Binding: Once binding to FMC63 was established, characterization of fusion protein binding to anti-CD19 expressing cells was to be investigated. Two cell types, HEK293T and T cells were utilized as binding targets.

(5a) In vitro anti-CD19 CAR-H Binding: To provide proof of concept that the fusion protein can successfully bind to anti-CD19 CAR expressing cells, HEK293T cells were transfected with the plasmid pSLCAR-CD19-28z, which contains an anti-CD19 CAR and GFP reporter via P2A. CAR-H cells were incubated with two different concentrations of fusion protein (136 or 13.6 ng/μL) at two different temperatures (Room Temp, or 37° C.). Transfection efficiency in this experiment was 84.60±1.06%, indicating that there was sufficient CAR+ cells to perform binding experiment.

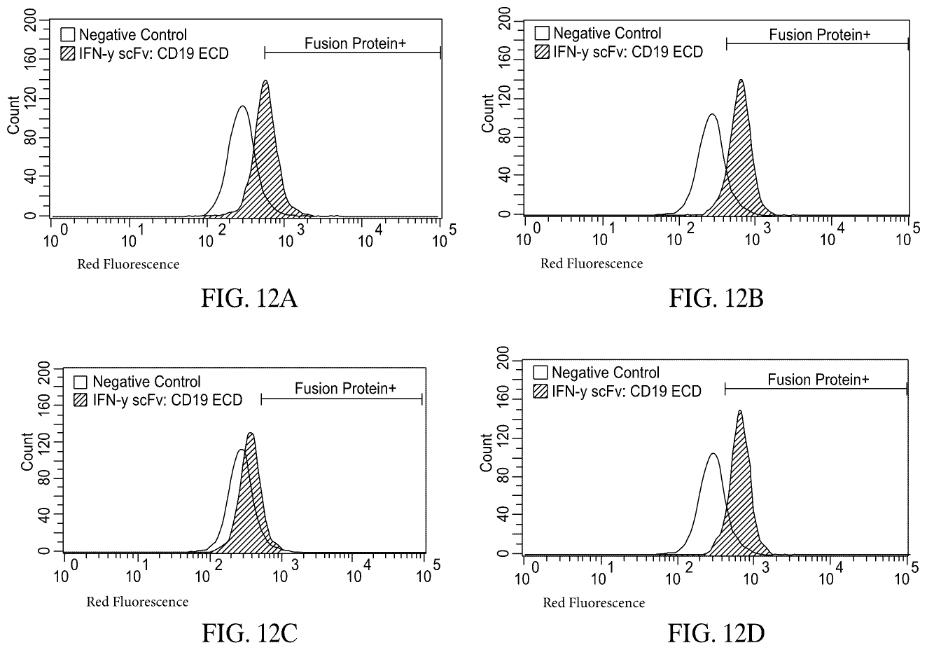

When comparing the histograms (FIG. 12 from the binding experiments, it is evident that incubating the fusion protein at room temperature promotes an increased binding to the CAR-H cells, which was seen at both fusion protein concentrations. Interestingly, at both binding temperatures, the lower concentration of fusion protein led to a higher percentage of binding to CAR-H cells. When binding at room temperature (FIG. 4a), incubating with 13.6 ng/μL fusion protein (FIG. 12A), led to binding to 96.45±0.04% of the CAR-H cells compared to 85.42±8.25% when incubating with 136 ng/μL (FIG. 12B). Similarly, when binding at 37° C., 13.7 ng/μL fusion protein bound to 87.66±2.54% of the CAR-H cells (FIG. 12C) compared to 22.04±0.31% when incubating with 136 ng/μL (FIG. 12D). Given the gating around GFP to isolate specific binding to the CAR-H; these results indicate that at a lower quantity of fusion protein, near complete capture of CAR-H cells is obtained. We hypothesize that at the higher concentration, aggregation may be occurring leading to a reduction in overall binding capacity and/or affinity of the fusion protein, but future experimentation may be performed to determine the binding curve and identify the maximum and minimum concentrations which promote binding.

(5b) In vitro anti-CD19 CAR-T Cell Binding: To further investigate fusion protein binding capabilities, binding of the fusion protein to CAR-T cells was evaluated in a comparable manner as CAR-H cells. Seven days post-transduction the anti-CD19 CAR expression was 63.20±1.57% via GFP, and gating was performed to analyze only the CAR+ population.

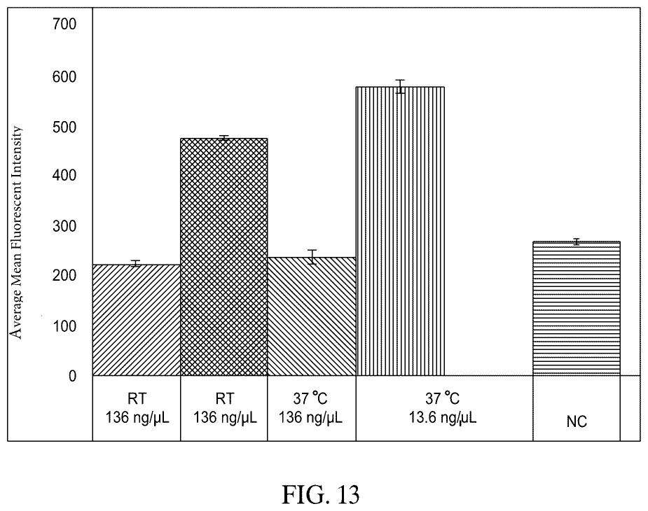

In similar fashion, to determine the impact of fusion protein concentration and binding temperature on CAR-T binding, two concentrations (136 or 13.6 ng/μL) at two different temperatures (Room Temp, or 37° C.) was evaluated (FIG. 13). At both the room temperature and 37° C. binding temperatures, there was a statistically significant difference between the high and low fusion protein binding (Room Temp p=<0.001; 37° C. p=0.008). There was a statistically significant binding of 13.6 ng/μL fusion protein at room temperature (M=478.49, SD=6.78); t(1.36)=−12.237, p=0.023 as well as at 37° C. (M=580.56, SD=29.32); t(1.51)=−9.29, p=0.0256 compared to the negative control (M=272.55, SD=15.44). Neither of the higher fusion protein concentrations had a statistically significant binding compared to the negative control. This result is interesting as it was hypothesized that the higher concentration would lead to an increase in binding to the CAR-T cells. However, we hypothesize that there may be issues with aggregation with the higher protein concentration, leading to a decrease in active binding sites and therefore a reduced binding capacity.

To determine the impact of binding temperature during fusion protein incubation, two different temperatures (Room Temperature and 37° C.) were evaluated. Since only the lower fusion protein concentration leads to any noticeable binding, only the lower concentration results will be discussed here. There was a higher binding at 37° C. (MFI=580.58, SD=29.32) than at room temperature (478.48, SD=6.68) however this difference was not statistically significant; t (1.1035)=−3.39, p=0.164. These results indicate that the fusion protein can specifically bind to CAR-T cells in a statistically significant fashion, with room to elucidate minimum and maximum binding properties.

Interestingly, the trend observed with the CAR-T cells with regards to binding temperature is opposite of what was observed with the CAR-H cells. With the CAR-H cells, room temperature binding promoted an increase in binding across all other conditions whereas here the opposite is true. In a similar fashion, there was a higher percentage of CAR+ cells captured with the CAR-H cells than the CAR-T cells across all conditions observed. We hypothesize that on each individual CAR+, there may be an increased density of CAR proteins on the surface of the CAR-H cells than the CAR-T cells which would explain these results in combination. If there are more CAR molecules present, then more fusion protein could bind at a lower temperature- and if you can bind more protein then naturally more of the CAR cells can be captured.

These results indicate that the fusion protein can specifically bind to CAR-T cells in a statistically significant fashion, with room to elucidate minimum and maximum binding properties.

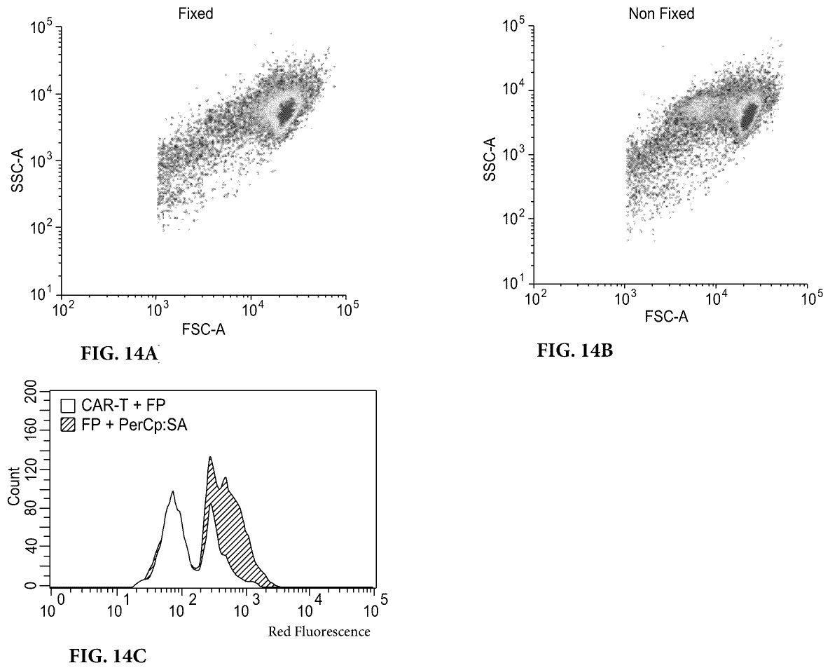

(5c) Dynamic Binding Interaction Evaluation: We hypothesized that some of the reduced binding of the fusion protein to CAR-T cells was due to activation of the CAR-T cells via binding to the anti-CD19 scFv present. To evaluate this, we performed a binding experiment with or without fixing the cells in between binding of the fusion protein, and the secondary PerCp-Streptavidin incubation (FIG. 14) As evident in FIG. 14, fixed cells (FIG. 14(A)) were much more tightly clustered in the same FSC vs SSC region however the unfixed cells (FIG. 14(B)) showed a clear second population with a reduced FSC. There was an observed dynamic interaction when binding the fusion protein to unfixed CAR-T cells leading to activation, down modulation and in some cases, we hypothesize internalization of the CAR complex.

To further understand the dynamic binding interaction, two methods of binding were evaluated. First, a standard staining procedure where the IFN-γ scFv: CD19 ECD fusion protein was incubated with fixed CAR-T cells initially, with secondary PerCp-Streptavidin staining following or where the fusion protein was incubated initially with the PerCp-Stretpavidin initially and then incubated with the CAR-T cells (FIG. 14(c)). With the ratio of fusion protein:streptavidin constant, and the timing of each incubation constant there was an observed dramatic increase in the binding when the fusion protein was initially incubated with the PerCp-Streptavidin than when the fusion protein was initially incubated with the CAR-T cells. This is due to avidity affects which mitigate a fast off-rate of the fusion protein CD19 ECD when bound to the CAR scFv. Initial incubation with streptavidin leverages the increased avidity due to the multiple biotin binding sides allowing for an increase in binding to the CAR-T cells.

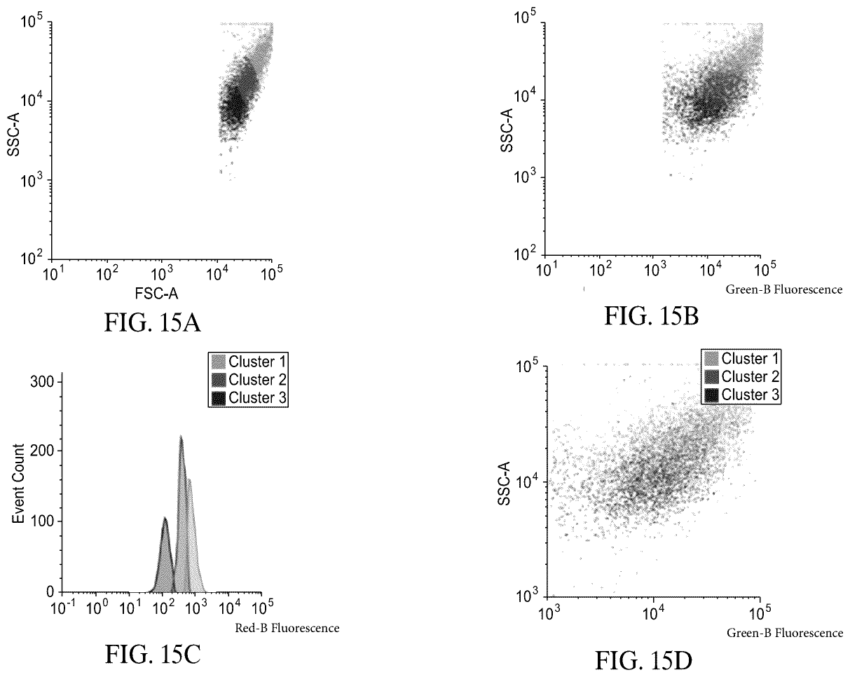

(5d) anti-CD19 CAR Selection: Building off the results from the previous section, fusion protein selection of anti-CD19 CAR-T cells based on CAR expression was evaluated. As evident in FIG. 15, there is a positive relationship between increases in FSC and SSC (FIG. 15A) with CAR expression (FIG. 15B). One side effect of having a cross-linked fusion protein is there is a crosslinking and stabilization of CAR+ cells when the fusion protein binds. Taking this a step further, K-means clustering was performed on the fusion protein binding following histogram (FIG. 15C) following initial incubation of the fusion protein with the streptavidin to identify three clusters of varying levels of fusion protein binding (FIG. 15C). The green fluorescence (i.e., CAR expression) of each of these clusters was plotted revealing a positive relationship between increased fusion protein binding and increased CAR expression.

These clusters of CAR+ cells increase the likelihood of a kinetic reaction, and there is evidence that in addition to leveraging avidity affects from the streptavidin, the binding reaction also leverages avidity affects from CAR clusters on the cells. As there are more CAR proteins on the cell surface, it is more likely that a stable complex is formed. This is evidence that the fusion protein can successfully form a stable binding complex with the CAR-T cells and that there is a way to isolate CAR-T cells based on CAR expression which may have important clinical relevancy.

### Discussion

As demonstrated herein, the inventors have successfully developed a novel fusion protein containing an IFN-γ scFv fused to a human CD19 extracellular domain suitable for identifying, and isolating CAR-T cells based on their cytokine secretion levels. The novel fusion protein utilizes an IFN-γ scFv derived from the therapeutic mAb, emapalumab (Gamifant, Novimmune SA) which has an extremely high affinity (picomolar range) to both free and receptor bound IFN-γ, and is an inhibitor of IFN-γ function, and accordingly capturing IFN-γ does not subject the cells to extensive IFN-472 γ stimulation.

Characterization of the fusion protein structure included SDS-PAGE, and multiple ELISAs to evaluate binding properties of each domain independently. Size evaluation of the fusion proteins IFN-γ scFv:CD19 ECD and CD19 ECD: IFN-γ scFv confirmed the expected molecular masses of 59 kDa and 58 kDa respectively. Further, both orientations of fusion proteins were treated with deglycosylation enzymes to confirm N-linked glycosylation pattern on both orientations.

Sandwich ELISAs were used to assess the binding of the fusion proteins to recombinant IFN-γ which revealed binding to IFN-γ in a dose-dependent manner with one exception. At 5 ng/uL IFN-γ the IFN-γ scFv:CD19 ECD had substantially higher binding than the reverse orientation fusion protein. Similar results were shown with the competitive binding assays where there was little binding of the CD19 ECD: IFN-γ scFv fusion protein in the presence of the competitor which was performed using 5 ng/uL of each protein. Despite this difference, these results present evidence that the IFN-γ scFv is functionally active in both orientations of fusion proteins and capture of soluble IFN-γ can be achieved using either orientation of novel fusion protein.

When evaluating the CD19 ECD functionality, capture via FMC63-Fc was utilized and the inventors demonstrated that both orientations of fusion protein showed statistically significant binding to surface bound FMC63-Fc demonstrating the ability of the fusion protein to be recognized and captured by the commonly utilized anti-CD19 scFv. This is a critical step in the characterization, as folding of the protein must allow not only the binding of the soluble IFN-γ, but ability to be recognized by anti-CD19 CAR expressing cells.

Two cell types were used to evaluate the in vitro binding of the fusion protein. Transient transfection of HEK293T cells with an anti-CD19 CAR was used as a proof-of-concept method. Initial binding results showed that the IFN-γ scFv: CD19 ECD fusion protein could bind to nearly 96% of all CAR-expressing HEK cells indicating that the fusion protein quantity was sufficient to saturate the CD19 binding sites. However, there were differences between the two concentrations of fusion protein utilized, with the higher quantity showing reduced binding. Despite this, we are confident that the fusion protein was binding to CAR-H cells specifically.

In vitro binding of the fusion protein was further evaluated with CAR-T cells which revealed lower binding to the CAR-T cells however there was statistically significant binding (via MFI) of both the higher and lower fusion protein concentrations to the CAR-T cells suggesting the specific binding was achieved.

Further investigation into the binding reaction indicated that stabilization of the binding reaction was necessary, which was achieved via fixation of the CAR-T cells prior to fusion protein incubation as well as forming initial fusion protein-streptavidin complexes prior to incubation with CAR-T cells.

By leveraging avidity effects on either side of the complex, through the multiple biotin binding sites on the streptavidin, and forming CAR clusters on the surface of the T cells, a stable binding complex capable of selection CAR-T cells based on the levels of CAR expression was developed.

We have developed and characterized a novel fusion protein which can bind to soluble IFN-γ and to CAR-expressing cells specifically and can isolate CAR-T cells based on CAR expression.

### Example 2

### Construction and Evaluation of TNF-α & CD19 Fusion Proteins

### Materials and Methods

(1) Construction of Fusion Protein Production Plasmids

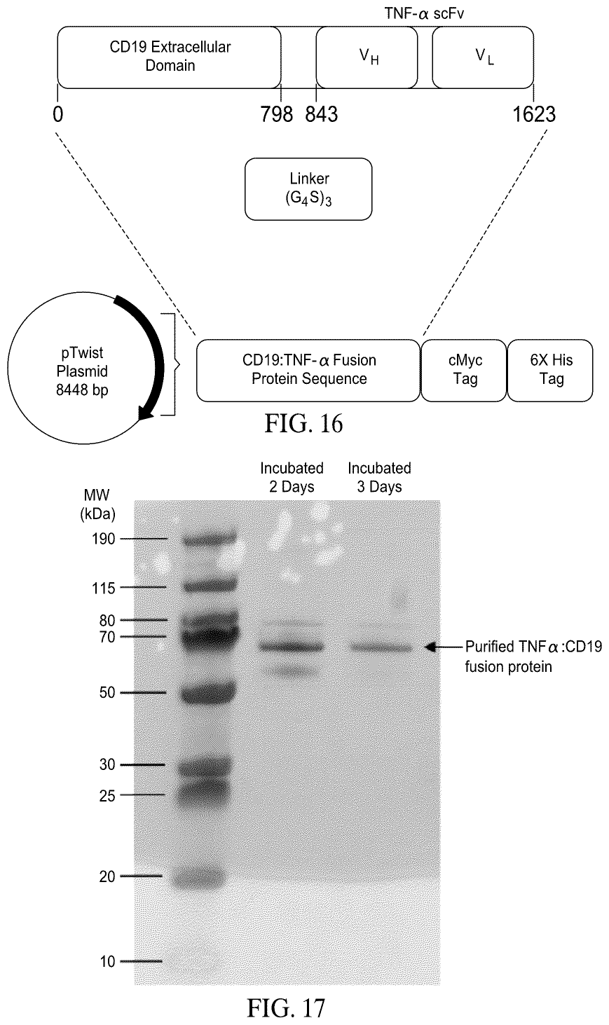

The TNF-α:CD19 ECD plasmid used a pTwist-CMV-BetaGlobin-WPRE-Neo vector from Twist BioSciences (San Francisco, CA). Twist Biosciences cloned fusion protein features in FIG. 16 using EcoRI and XhaI restriction sites, including Myc-tag, 6×-His tag, and IFN-γ scFv and CD19 extracellular domain linked via a flexible peptide (Gly4Ser)3 (SEQ ID NO:15) linker. The woodchuck hepatitis virus posttranscriptional regulator element (WPRE), Beta Globin vector, and CMV all enhance transgene expression. Combined, the plasmid successfully produced fusion protein when used for transfection and was named “ClonedTNFFP”.

(2) Cell Culture & (3) Transient Transfection

The HEK298 cells were passaged then added to 8 wells of a 24 well plate (Corning, Corning, NY), preferably wells away from the edges to avoid evaporation. They were then incubated overnight to reach 80% confluency with 1 mL total of media and harvested cells per well. 150,000 cells per well were added and media was Opti-MEM Reduced Serum Medium (ThermoFisher), supplemented with 0.2% 100 mM Sodium Pyruvate and 5% FBS. On day 2, Twist plasmid was removed from freezer and centrifuged until maximum speed reached, then stopped immediately. Tube of dry plasmid resuspended in 200 μL of TE buffer and pipetted up and down gently several times resulting in 50 ng/μL solution. Transfection was performed with Lipofectamine 3000 kit (ThermoFisher, Catalog #L300000) in the refrigerator. In Tube A, 1.5 μL Lipofectamine 3000 and 25 uL OptiMEM Reducing Serum Medium were added per well. In tube B, 500 ng of plasmid (10 μL/well), 25 uL OptiMEM Reducing Serum Media and 1 μL P3000 Reagent were added per well. The mixtures were combined, incubated for 15 minutes, and distributed to all 8 wells that were swirled gently on a table to distribute lipofectamine. Cells were then incubated at 37° C. and 5% CO₂. Three wells were harvested after 2 days incubation and three wells were harvested after 3 days incubation, each set having a control well. Harvest collected in tube labeled with date, contents, and concentration. Concentration found with TAKE3 plate in White 114, where 2 μL ultrapure water is pipetted with a 2 μL pipette on the first spot, followed by two 2 μL additions of well-mixed supernatant collected during harvest placed on open spots on the plate. Before and after adding samples to the TAKE3 plate, wipe it with a chem wipe to clean. Second transfection was achieved with the same protocol, except with one well in a 6 well plate. The increased surface area of a 6 well plate required a total of 2 mL/well media plus cells and an extra day to reach 80% confluency, so using a 6 well plate is not recommended. One trial evaporated overnight, so checking incubated cells everyday is necessary. Note, some pipette tips that fit a 20 μL pipette can only hold 10 μL of liquid.

(4) SDS-PAGE Gel Electrophoresis

The SDS-PAGE gel run was performed. A NuPAGE™ 4-12% Bis-Tris Mini Gel 1.0 mm (Invitrogen, Carlsbad, CA) was used, where the comb was gently removed, white strip was peeled off, the wells were gently washed with ultrapure water to remove bubbles, and the gel was placed in the apparatus so the low side was facing the back. In a 1 L container, add 50 mL of 20× transfer buffer (NP00061) to 950 mL of ultrapure water. Fill the back side of the gel up with this transfer buffer. To 600 mL of transfer buffer, add 1 μL of NuPAGE™ Antioxidant (Catalog #NP0005, Invitrogen, Carlsbad, CA) and add this mixture to the front side of the gel. Find and label one microtube per well. In each, add 1.5 pg of unfiltered fusion protein (volume of protein calculated based on concentration), 6.25 μL NuPAGE™ sample reducing agent (NP0004, Invitrogen), 1 μL NuPAGE™ Sample Reducing Buffer (Catalog #NP0008, Invitrogen, Carlsbad, CA), and the remaining volume ultrapure water for a total of 25 μL. microtubes were then heated at 70 C for 10 minutes to denature proteins. A 15 μL protein standard sample (found in the freezer, catalog #26619) was added to the first well of the gel and the supernatant samples from 2 and 3 days of incubation were added to the next two wells. Cycle ran for 30 minutes. Gel removed from apparatus and taken to Elmer's lab for staining. The gel was removed from plastic cover with a putty knife. Placed in old box previously used for holding pipette tips, where the box was filled with ultrapure water enough to cover the gel. Box placed on a tilting rotator for 5 minutes, then the water was dumped and replaced. This wash happened 3 times, then Coomassie blue staining was added so the gel was fully covered. After 1 hour tilting, the stain was poured out and 3 more washes were done with ultrapure water, tilting for 15 minutes each time. After this, the box with the gel was left tilting overnight in ultrapure water. The next day, photos of the gel were taken with a photocopier by the computer. Gel images are provided in Results.

(5) Biotinylation of the Fusion Protein

Biotinylation was performed with EZ-Link Micro Sulfo-NHSLC Biotinylation kit (ThermoFisher Scientific, Rockford, IL, Catalog #21935) according to the manufacturer's guidelines using Zeba Desalting column (ThermoFisher, Catalog #89889). Biotinylation quantitation was performed with Pierce™ Biotin Quantitation Kit (ThermoFisher, Catalog #28005). Buffer exchange was performed with Zeba Desalting spin column according to procedure. Both procedures are in the Appendix.

(6) Enzyme-Linked Immunosorbent Assay

The standard curve ELISA was performed with plate and reagents from a TNF-α ELISA kit (ThermoFisher, Catalog #KHC3011), where procedure was identical to ELISA procedure in the appendix for wells A1-6. In wells B1-6, the same procedure was followed except 100 μL/well of biotinylated fusion protein (500 ng/well) was added replacing the biotin conjugate.

A sandwich ELISA was performed, where 500 ng of fusion protein was added to the bottom of each well for 15 wells total and incubated overnight at 37° C. The following day, wells were blocked with addition of 100 μL/well non-fat dry milk (NFDM) buffer and incubated for 1 hour at room temperature. All reagents and buffers except Streptavidin-TRP were removed from the refrigerator so they would be at room temperature when added. Then, each different concentration of TNF-α protein was added to 3 wells and incubated for 2 hours at room temperature. Concentrations were prepared with TNF-α dilution buffer in the kit and were prepared 1000 pg/mL, 500 pg/mL, 250 pg/mL, 125 pg/mL, 62.5 pg/mL, and a PBS blank. Instructions provided with kit detail TNF-α dilutions. Then, 100 μL/well of anti-TNF-α conjugate protein that came with the kit was added and incubated for an hour. Streptavidin-HRP was diluted according to kit specifications with SAV diluent and 100 μL/well were added and incubated at room temperature for 30 minutes. Then 100 μL/well TMB was added in a dark room so that the buffer was not exposed to light and incubated 30 minutes. Finally, 100 μL/well stopping solution was added and the wells were analyzed with a plate reader at 450 nm. After each incubation step except TMB incubation, the wells were emptied and washed 3 times with 100 μL ELISA wash buffer (PBSt).

The binding assay without capture antibody had 500 ng/well of TNF-α plated overnight. Recombinant human TNF-α (R&D Systems, Catalog #210-TA/CF) reconstituted in PBS resulting in 0.5 ug/μL solution, stored at −20° C. The next day, plate blocked with 100 μL/well non-fat dry milk (NFDM) buffer. Biotinylated fusion protein was then added to wells in the following dilutions: 500, 50, 5, 0.5, and 0.05 ng/well. Streptavidin-RP concentrate stock from the TNF-α ELISA kit was depleted so subsequent ELISA trails used different HRP-streptavidin stock (ThermoFisher 1.25 mg/mL catalog #N100) in a 1:8000 dilution with SAV diluent previously used in ELISA kit. Incubated at room temperature for 30 minutes. Then 100 μL/well TMB was added in a dark room so that the buffer was not exposed to light and incubated 15 minutes. Finally, 100 μL/well stopping solution was added and the wells were analyzed with a plate reader at 450 nm. After each incubation step except TMB incubation, the wells were emptied and washed 3 times with 100 μL/well ELISA wash buffer (PBSt). Note, incubation of TMB was 15 rather than the recommended 30 minutes to prevent over exposure.

The competitive binding assay had 500 ng/well TNF-α plated overnight and the next day blocked with 100 μL/well NFDM buffer. After washing, three wells had PBS added and three wells had anti-TNF-α mAb added. Incubated 1 hour. Then, a master mix was prepared with 3000 ng biotinylated CD19:TNF-α fusion protein diluted with PBS. 100 μL master mix was added to each well resulting in 500 ng/well. Subsequent steps same as above with 20 minute TMB incubation to prevent overexposure. All steps were incubated at 4° C. except TMB incubation which was incubated at room temperature.

The FMC63 binding assay had 500 ng/well FMC63 plated overnight and the next day blocked with 100 μL/well NFDM buffer. After washing, 100 μL/well of the following were added: three wells with PBS, three wells with 0.5 ng/well CD19:TNF-α fusion protein, and three wells with 500 ng/well biotinylated CD19:TNF-α fusion protein. PBS used as dilution buffer and incubated 1 hour. Subsequent steps same as above with 15 minute TMB incubation. All steps were incubated at 4° C. except TMB incubation, which was incubated at room temperature.

Synthesizing PBS was accomplished by mixing 1000 mL ultrapure water (lab next to 216) and PBS dry mix packets (under the lab bench in second cabinet from the right). This is typically mixed in glass jars with an orange plastic lid and autoclaved. If using PBS for nonsterile applications, aliquot it into a separate 50 mL tube to reduce contamination of the autoclaved batch. This is something to keep in mind for buffers in general. PBSt was made adding 0.05 volume % Tweene20 (in refrigerator if not on lab bench) to PBS and was used as ELISA wash buffer. For 30 mL batch, 0.015 mL Tween20 was added to 29.95 mL PBS. Non-fat dry milk blocking buffer was made where it was 5 mass % non-fat dry milk (box in the same cabinet as PBS dry packets) dissolved in PBSt. So, 30 mL of PBSt would have 1.5 g of non-fat dry milk powder added.

Results

Producing TNF-α:CD19 fusion protein was a success, with concentrations of protein in harvest supernatant in Tables 1 and 2.

TABLE 1
Harvest results after first transfection of fusion protein.
Concentration of protein and 260/280 ratio of supernatant of
transfected HEK298 cells after 2 and 3 days of incubation.

| Days of HEK293 | Concentration of | |
|---|---|---|
| Cell Incubation | Protein (mg/mL) | 260/280 |
| Day 2 | 3.72 | 0.71 |
| Day 3 | 4.2 | 0.729 |

TABLE 2
Harvest results after second transfection of fusion protein.
Concentration of protein and 260/280 ratio of supernatant
of transfected HEK298 cells.

| | Concentration of | |
|---|---|---|
| Protein | Protein (mg/mL) | 260/280 |
| Fusion Protein | 0.8 | 0.83 |
| FMC63 | 1.4 | 0.89 |

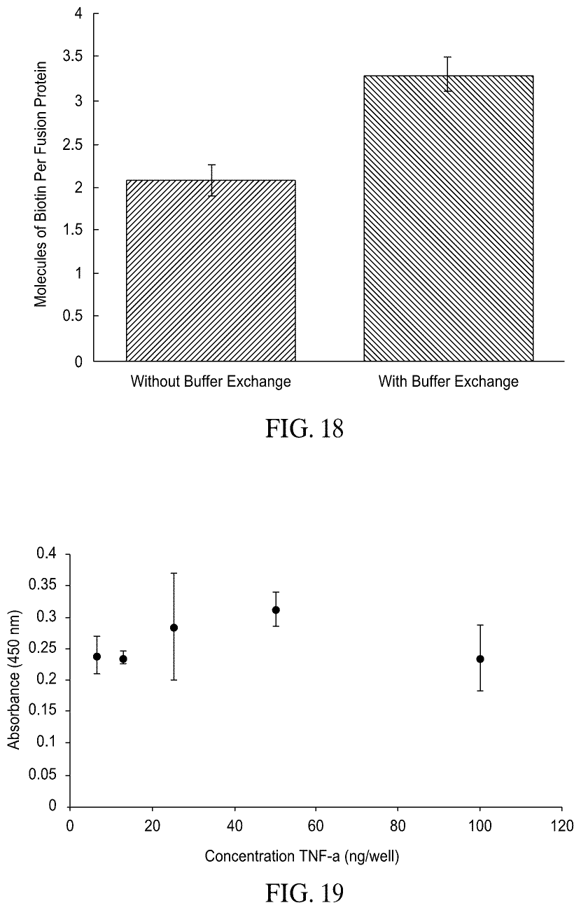

Both transfections were successful. As expected with the first transfection, more protein was produced after longer incubation. The protein purity was acceptable for both, where the 260/280 ratio indicated little nucleic acid contamination. For the first transfection, testing the prevalence of fusion protein in the supernatant was done with an SDS-PAGE electrophoresis gel without filtration because the imidazole to elute the His-tag from the Ni resin in chromatography has the same absorbance of fusion protein, rendering the exact concentration of protein indistinguishable. The gel with unfiltered supernatant after 2 and 3 days of incubation is FIG. 17.

The darkest band in the gel corresponds to the 61,967 Da molecular weight of TNF-α:CD19 fusion protein, suggesting it was substantially more prevalent than other proteins in the sample. Because the bands above and below the fusion protein were so weak relatively, it was not suspected that the other proteins would obstruct the fusion protein binding during characterization. The presence of other proteins did, however, affect biotinylating, as nitrogenous bases in the supernatant prevented biotin binding to fusion protein. Performing a buffer exchange using a Zeba desalting column to remove nitrogenous bases improved the molecules of biotin per molecule of fusion protein from 2.1 to 3.3, as determined with the biotinylation quantitation kit. Results shown in FIG. 18.

Biotinylation concerns were not addressed for initial failed ELISA variations, which implies they could have contributed. The first of these attempted ELISA's was a sandwich ELISA, shown in FIG. 19.

Over the course of a 100-fold TNF-α dilution, absorbance does not have an increasing trend. This shows no binding occurrence despite decent absorbance because empty wells still have some absorbance, so absorbance trends are more important than absorbance magnitude. The standard curve ELISA was later performed with the biotin conjugate as a detection antibody in half of the wells to obtain a typical binding curve.

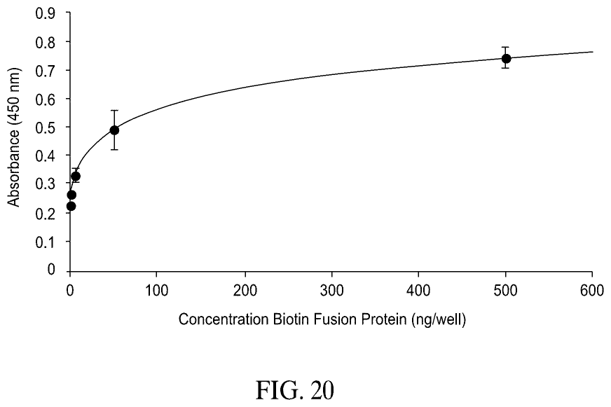

Negligible increase in absorbance with increase fusion protein addition relative to a biotinylated anti-TNF-α antibody was observed. One possible explanation was that the capture antibody was already bound to an epitope the fusion protein was targeting. Antibodies have two scFv's, so they have a binding advantage over the fusion protein. Not to mention steric hindrance also could have prevented the fusion protein from binding. For this reason, the capture antibody was removed and 500 ng/well TNF-α was plated on the bottom of the well. Results shown in FIG. 20.

This was the first successful demonstration of binding, where a sigmoidal curve characteristic of such binding was demonstrated. Achieving this after removing the capture antibody suggests it is possible the capture body interfered with previous ELISA trials. It is important to note that TNF-α and fusion protein concentration changes could have improved results, as in previous ELISA variations, recommended TNF-α concentrations were in pg/mL, making it a limiting reagent. In this variation, 500 ng/well of both TNF-α and fusion protein were added, no longer making TNF-α a limiting reagent. This abundance of both TNF-α and fusion protein became obvious when overexposure occurred during initial trials and is therefore likely the reason for better absorbance values. This does not, however, disqualify this demonstration of fusion protein binding, as absorbance trends, rather than magnitude, demonstrate binding.

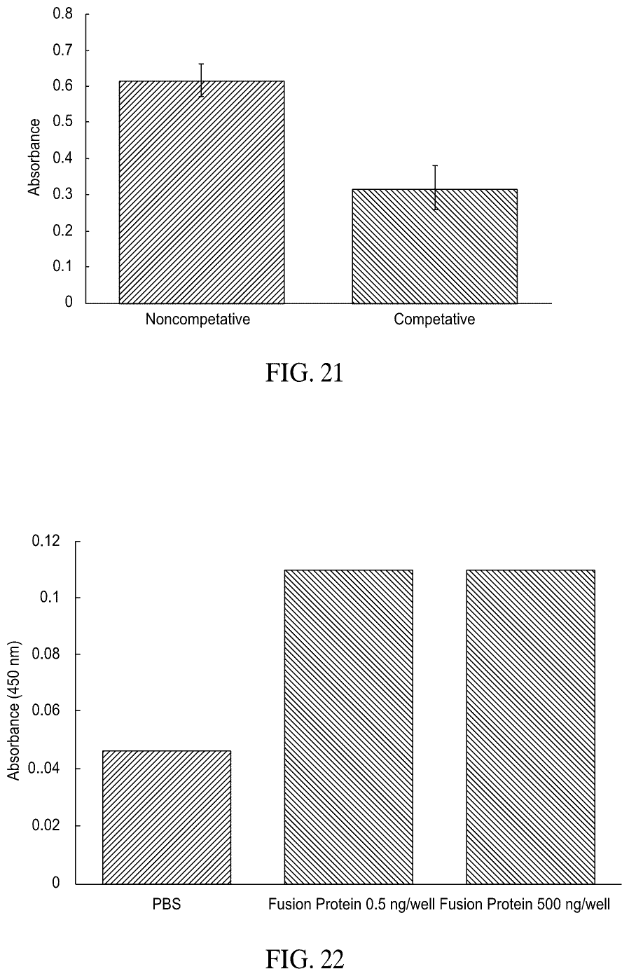

Next, a competition ELISA was performed to see the effects of fusion protein binding capacity when an anti-TNF-α mAb was already bound to TNF-α. Once again, TNF-α coated the bottom of the well to prevent interference from other antibodies. Results shown in FIG. 21.

Results show a 48.4% decrease in absorbance when the fusion protein had to compete with an anti-TNF-α mAb. This result suggests the scFv of the fusion protein had an affinity for TNF-α that was stronger than steric hinderance effects or even strong enough to remove anti-TNF-α mAb from its epitope.

Characterizing the CD19 side of the molecule required use of FMC63 protein as the target antigen. In these ELISA variations, it coated the bottom of the wells. The first attempts resulted in similar absorbance to wells without fusion protein addition, which confirmed that no binding occurred. The results are shown in FIG. 22.

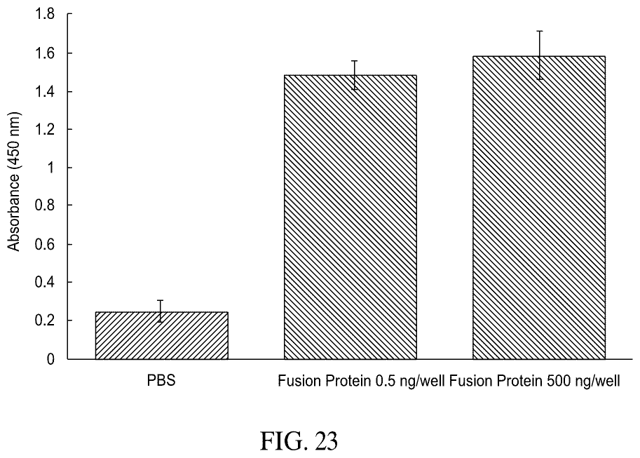

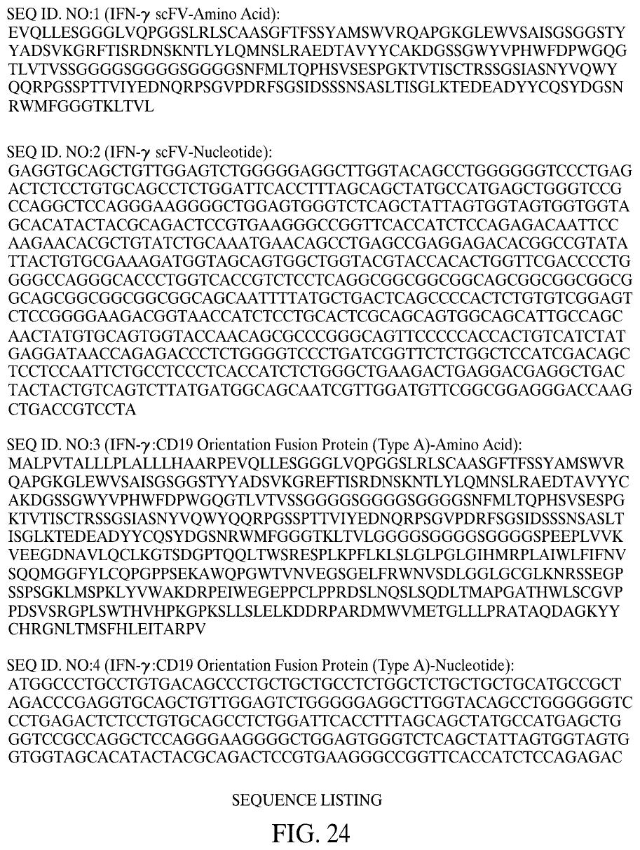

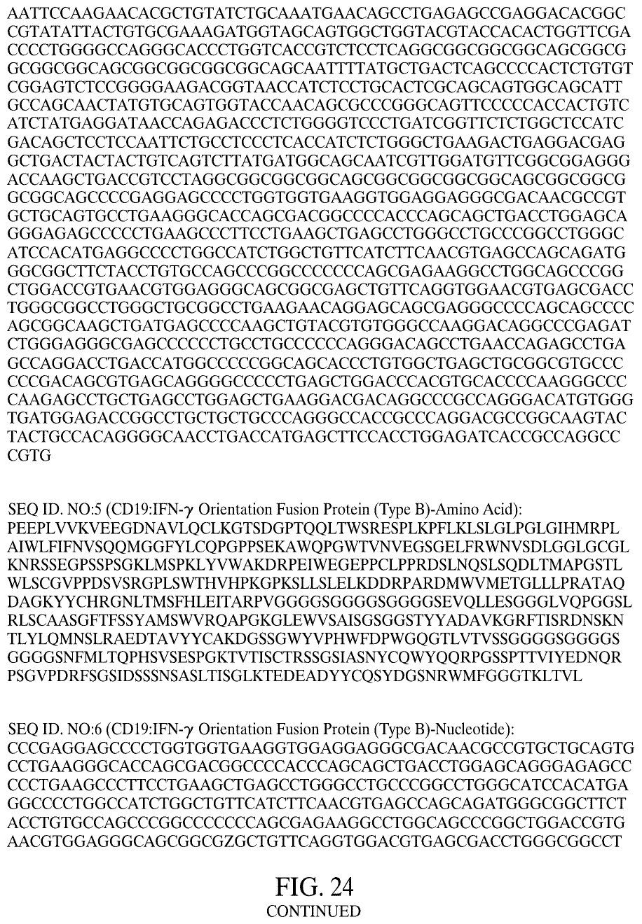

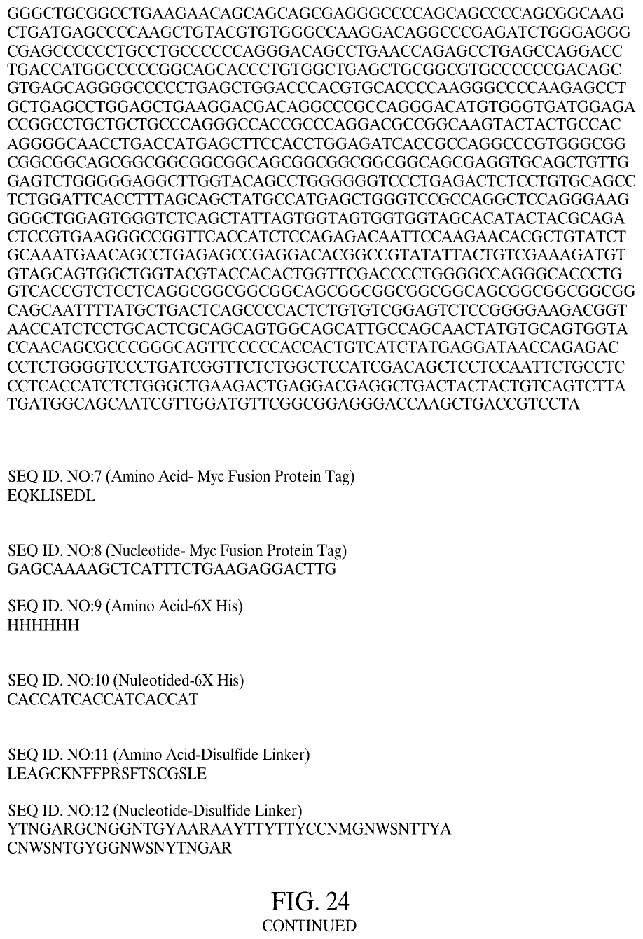

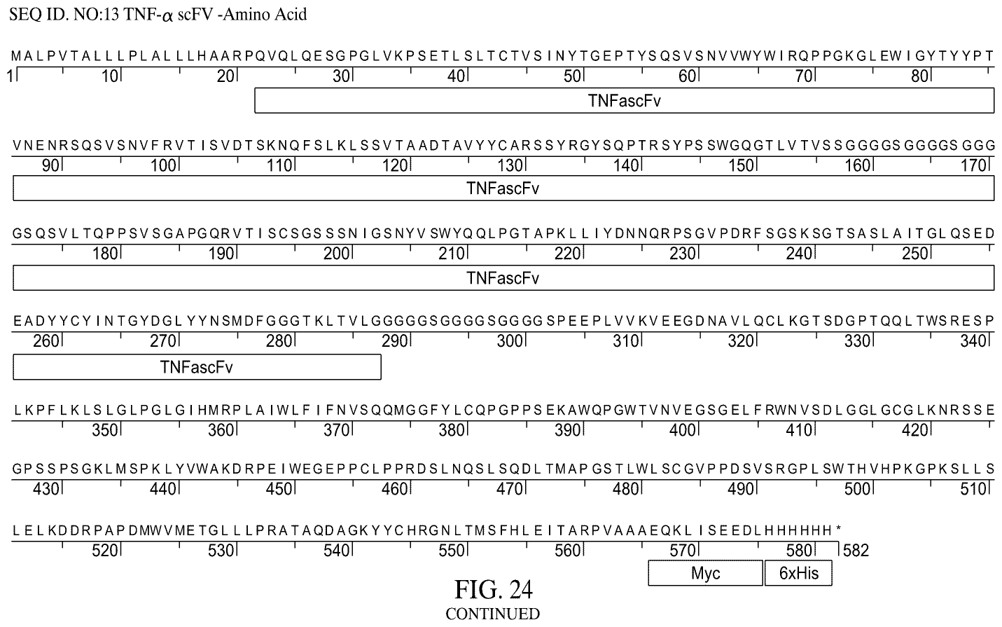

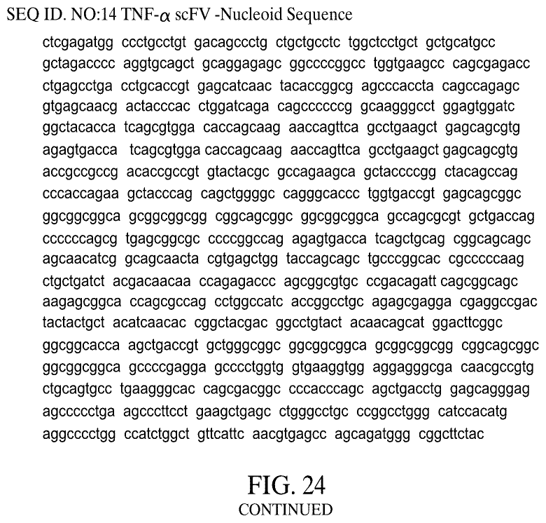

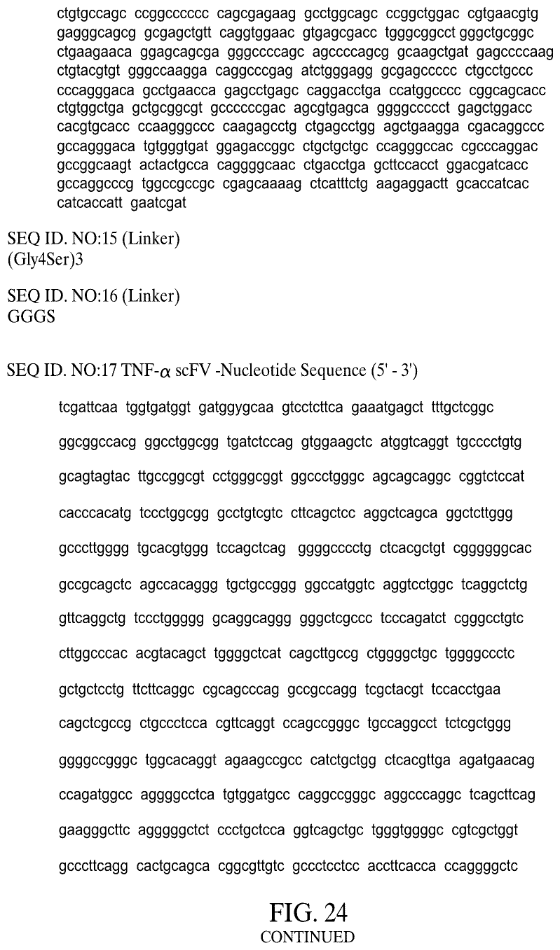

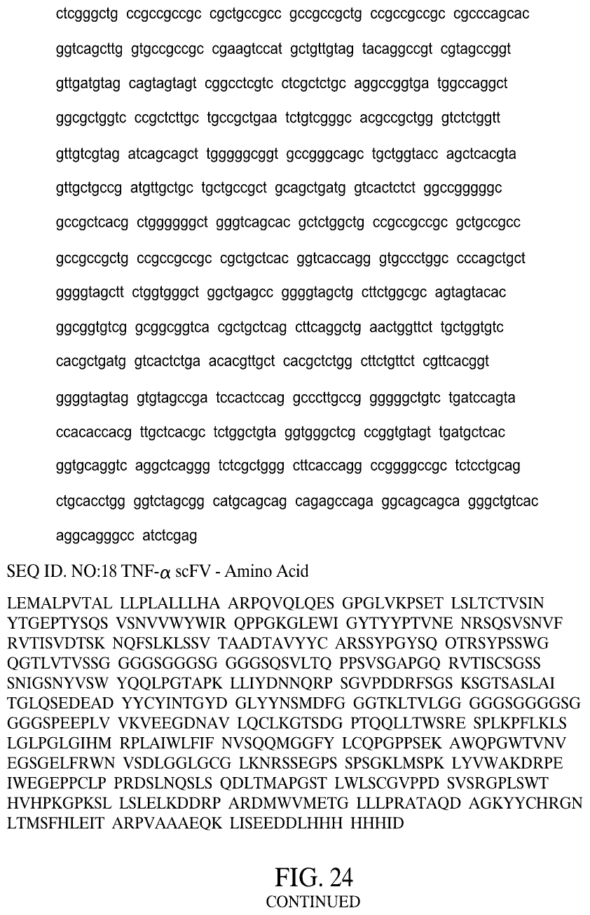

The lack of demonstrated binding prompted a new batch of FMC63 and fusion protein produced with HEK293 cell transfection, in hopes that the fusion protein would fold correctly and bind better. The results of repeating the same ELISA with fresh fusion protein and FMC63 are shown in FIG. 23. Note, biotinylating this batch of fusion protein resulted in the best biotinylation results, 3.3 moles of biotin per mole of fusion protein.

Results were much improved, where absorbance increased over seven-fold when fusion protein was added. The CD19 arm of the fusion protein appears to have successfully bound to FMC63 in both concentrations. It was expected, however, that the disparity in absorbance values of the lower and higher concentration of fusion protein would have been larger. Perhaps 0.5 ng/well is near the saturation point for the fusion protein. Also, incubation was at 4° C. rather than room temperature to decrease the rate of dissociation.

### Discussion

Production, purification, and biotinylation of fusion protein was evidently one of the most important aspects of successful binding in ELISA assays. The choice to not separate fusion protein from supernatant via affinity chromatography appeared to be an acceptable approach from electrophoresis results. Without separation, however, protein concentration as determined with the TAKE3 plate reflects all supernatant proteins, implying the amount of fusion protein added was less than if the sample was pure fusion protein. Buffer exchanges removed many contaminants in supernatant, which decreased this effect resulting in a more accurate estimate of fusion protein concentration. Not to mention effective biotinylation improved absorbance values. The most impactful effect, however, was performing ELISA tests with fusion protein that had been produced that week. This showed vastly improved binding for both binding sites, suggesting either the first transfection resulted in fusion proteins that had misfolded, or over the course of six months degradation occurred. All supernatant samples were stored at 4° C., so storing at −20° C. will be considered in the future.

The presence of antibodies in ELISA variations also influenced the TNF-α binding capabilities of the fusion protein anti-TNF-α scFv. This was expected because antibodies have a binding advantage with two scFv's. Also, epitopes were unknown and could have been the same or in close proximity. Finally, the molecular weight of TNF-α is 17.3 kDa, which nearly a third of the 67 kDa fusion protein and a seventh of the average molecular weight of a monoclonal antibody, 150 kDa. Even if the anti-TNF-α scFv bound to a different epitope than an antibody, the larger size of both relative to the smaller antigen could result in steric hindrance. Improvement from the standard curve assay to the binding assay without capture antibodies demonstrated these effects, especially since the same batch of biotinylated fusion protein was used for both. The competitive binding assay relied on this concept, showing a 48.4% decrease in binding when anti-TNF-α mAb was added preceding the addition of fusion protein. Its results suggest scFv of the fusion protein could have had an affinity for TNF-α that was stronger than some steric hinderance effects or even strong enough to remove anti-TNF-α mAb from its epitope. Given that fusion protein will likely not have to compete with other binding molecules for soluble TNF-α from live CAR T cells, binding capabilities of the TNF-α scFv are very adequate for a functional fusion protein.

FMC63 is an antibody with an anti-CD19 scFv used in CAR T therapies such as Kymriah, so it was ideal to mimic binding of fusion protein CD19 extracellular domain to an anti-CD19 CAR. Binding occurred when 0.5 ng/well and 500 ng/well of fusion protein was added to wells as evidenced by a sevenfold increase in absorbance over wells with no fusion protein addition. Relative to each other, the two concentrations did not have a significant difference in absorbance (p=0.4058), which was unexpected considering the concentrations are 1:1000 ratio. This likely occurred because 0.5 ng/well was enough to saturate FMC63 and excess fusion protein was washed away. Both FMC63 and fusion protein were reproduced using HEK293 cells the week of the successful ELISA they were used in, so failures of earlier FMC63 ELISA trials were concluded to have been caused by protein misfolding or denaturation over a period of 4 months.

### Example 3

### Fusion Protein, in the Presence of IFN, Enhances Bead-Mediated Isolation of CAR+ Cells

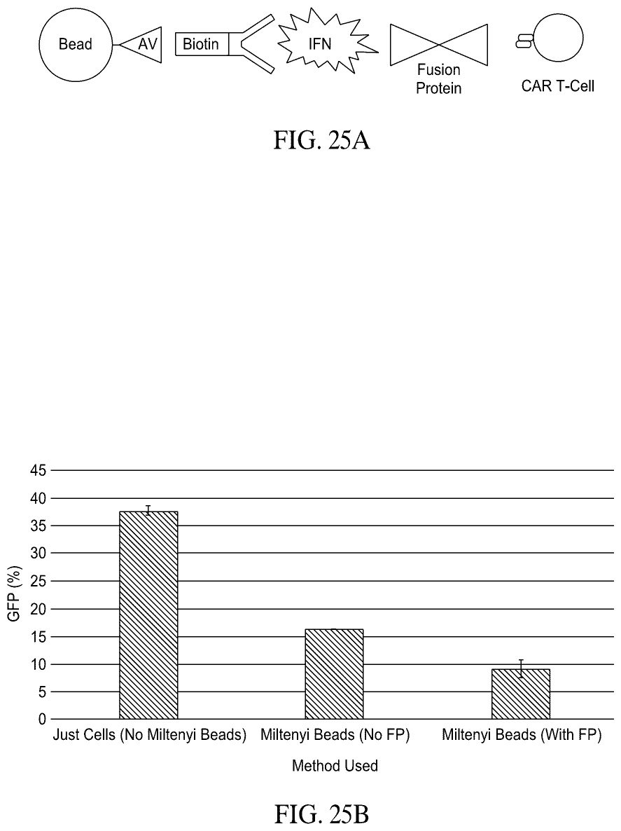

An experiment was conducted to assess the binding of CAR+ cells from CART populations to beads via the complex shown in FIG. 25A. The beads used in this experiment were Miltenyi's (Miltenyi Biotec, Germany) magnetic microbeads, labeled with avidin. An anti-IFN biotynlated antibody was attached to this bead and then a 1.5× excess of IFN was added to the solution. As per FIG. 25B, the cell population was measured by flow cytometry to be originally 38% CAR+ (as indicated by % GFP being expressed) before being exposed to the beads. As an experimental control, this CAR+ population was exposed to the complex in FIG. 25A without the fusion protein for 60 minutes at room temperature and then the beads were removed with the MACS magnetic separator. Flow cytometry results showed that only 17% of the CAR+ cells remained in the supernatant, indicating that a significant number of CAR+ cells were removed via non-specific binding to the beads (presumably by attachment to the bead surface or the exposed IFN on the bead surface). The same procedure was followed when the full complex from FIG. 25A (i.e. including the IFN fusion protein) was allowed to bind to the cells (i.e. for 60 min at room temperature). The Flow results here showed that only 9% of the CAR+ cells remained in the supernatant, indicating that some of the CAR+ cells did bind to the beads via the full complex in FIG. 25A. Note: the two experimental conditions (ie the “just cells” and the “with Fusion Protein” conditions) were run in triplicate, the cell concentration for binding experiments was 1×10\hat{ }6 cells/ml and the fusion protein concentration used was 1 ng/ul.

Future experiments will be run with BIG beads (labeled with avidin) that can be used in scaleable chromatography experiments to reduce the amount of non-specific binding seen here and maximize the amount of binding to the fusion protein via the complex shown in FIG. 25A, importantly, this experiment shows that the fusion protein attached to beads can bind CAR+ T cells in the presence of IFN. Additional experiments are in process for developing cloned populations of cells that secrete varying levels of IFN, to show that the complex in FIG. 25A can isolate CAR+ cells based on the degree to which they secrete IFN.

The following references listed below are incorporated herein by references:

### REFERENCES

1. Maude, S. L.; Laetsch, T. W.; Buechner, J.; Rives, S.; Boyer, M.; Bittencourt, H.; Bader, P.; Verneris, M. R.; Stefanski, H. E.; Myers, G. D.; et al. Tisagenlecleucel in Children and Young Adults with B-Cell Lymphoblastic Leukemia. New England Journal of Medicine 2018, 378, 439-448, doi:10.1056/nejmoal709866.
2. Schuster, S. J.; Bishop, M. R.; Tam, C. S.; Waller, E. K.; Borchmann, P.; McGuirk, J. P.; Jäger, U.; Jaglowski, S.; Andreadis, C.; Westin, J. R.; et al. Tisagenlecleucel in Adult Relapsed or Refractory Diffuse Large B-Cell Lymphoma. New England Journal of Medicine 2019, 380, 45-56, doi:10.1056/nejmoal804980.
3. Neelapu, S. S.; Locke, F. L.; Bartlett, N. L.; Lekakis, L. J.; Miklos, D. B.; Jacobson, C. A.; Braunschweig, I.; Oluwole, O. O.; Siddiqi, T.; Lin, Y.; et al. Axicabtagene Ciloleucel CAR T-Cell Therapy in Refractory Large B-Cell Lymphoma. New England Journal of Medicine 2017, 377, 2531-2544, doi:10.1056/nejmoal707447.
4. Davila, M. L.; Riviere, I.; Wang, X.; Bartido, S.; Park, J.; Curran, K.; Chung, S. S.; Stefanski, J.; Borquez-Ojeda, O.; Olszewska, M.; et al. Efficacy and Toxicity Management of 19-28z CAR T Cell Therapy in B Cell Acute Lymphoblastic Leukemia. Science Translational Medicine 2014, 6, 224ra25-224ra25, doi:10.1126/scitranslmed.3008226.
5. Xhangolli, I.; Dura, B.; Lee, G. H.; Kim, D.; Xiao, Y.; Fan, R. Single-Cell Analysis of CAR-T Cell Activation Reveals A Mixed TH1/TH2 Response Independent of Differentiation. Genomics, Proteomics and Bioinformatics 2019, 17, 129-139, doi:10.1016/j.gpb.2019.03.002.
6. Stock, S.; Schmitt, M.; Sellner, L. Optimizing Manufacturing Protocols of Chimeric Antigen Receptor t Cells for Improved Anticancer Immunotherapy. International Journal of Molecular Sciences 2019, 20, doi:10.3390/ijms20246223.
7. Graham, C.; Jozwik, A.; Pepper, A.; Benjamin, R. Allogeneic Car-t Cells: More than Ease of Access? Cells 2018, 7, 155, doi:10.3390/cells7100155.
8. Global T-Cell (CAR-T, TCR, and TIL) Therapy Market (4th Edition), 2019-2030|Industry Analysis|Market Size|2030 Available online: https://www.rootsanalysis.com/reports/view_document/t-cell-immunotherapies-market-4th-edition-2019-2030/261.html (accessed on 26 May 2020).
9. AlloSHRINK—Standard CHemotherapy Regimen and Immunotherapy With Allogeneic NKG2D-Based CYAD-101 Chimeric Antigen Receptor T-Cells—Full Text View—ClinicalTrials.Gov Available online: https://www.clini-caltrials.gov/ct2/show/NCT03692429?term=NCT03692429&draw=2&rank=1 (accessed on 26 May 2020).
10. Safety and Efficacy of ALLO-501 Anti-CD19 Allogeneic CAR T Cells in Adults With Relapsed/Refractory Large B Cell or Follicular Lymphoma—Full Text View—ClinicalTrials.Gov Available online: https://clinicaltri-als.gov/ct2/show/NCT03939026 (accessed on 26 May 2020).
11. Schubert, M. L.; Huckelhoven, A.; Hoffmann, J. M.; Schmitt, A.; Wuchter, P.; Sellner, L.; Hofmann, S.; Ho, A. D.; Dreger, P.; Schmitt, M. Chimeric Antigen Receptor T Cell Therapy Targeting CD19-Positive Leukemia and Lymphoma in the Context of Stem Cell Transplantation. Human Gene Therapy 2016, 27, 758-771, doi:10.1089/hum.2016.097.
12. Turtle, C. J.; Hanafi, L. A.; Berger, C.; Gooley, T. A.; Cherian, S.; Hudecek, M.; Sommermeyer, D.; Melville, K.; Pender, B.; Budiarto, T. M.; et al. CD19 CAR-T Cells of Defined CD4+:CD8+ Composition in Adult B Cell ALL Patients. Journal of Clinical Investigation 2016, 126, 2123-2138, doi:10.1172/JC185309.
13. Zhang, H.; Zhao, P.; Huang, H. Engineering Better Chimeric Antigen Receptor T Cells. Experimental Hematology and Oncology 2020, 9, 1-13, doi:10.1186/s40164-020-00190-2.
14. Eyquem, J.; Mansilla-Soto, J.; Giavridis, T.; van der Stegen, S. J. C.; Hamieh, M.; Cunanan, K. M.; Odak, A.; Gonen, M.; Sadelain, M. Targeting a CAR to the TRAC Locus with CRISPR/Cas9 Enhances Tumour Rejection. Nature 2017, 543, 113-117, doi:10.1038/nature21405.
15. Schmueck-Henneresse, M.; Omer, B.; Shum, T.; Tashiro, H.; Mamonkin, M.; Lapteva, N.; Sharma, S.; Rollins, L.; Dotti, G.; Reinke, P.; et al. Comprehensive Approach for Identifying the T Cell Subset Origin of CD3 and CD28 Antibody-Activated Chimeric Antigen Receptor-Modified T Cells. The Journal of Immunology 2017, 199, 348-362, doi:10.4049/jimmunol.1601494.
16. Bai, Z.; Woodhouse, S.; Zhao, Z.; Arya, R.; Govek, K.; Kim, D.; Lundh, S.; Baysoy, A.; Sun, H.; Deng, Y.; et al. Single-Cell Antigen-Specific Landscape of CAR T Infusion Product Identifies Determinants of CD19-Positive Relapse in Patients with ALL. Science Advances 2022, 8, doi:10.1126/sciadv.abj2820.
17. Bhat, P.; Leggatt, G.; Waterhouse, N.; Frazer, I. H. Interferon-7 Derived from Cytotoxic Lymphocytes Directly Enhances Their Motility and Cytotoxicity. Cell Death and Disease 2017, 8, e2836, doi:10.1038/CDDIS.2017.67.
18. McKenzie, M. D.; Dudek, N. L.; Mariana, L.; Chong, M. M. W.; Trapani, J. A.; Kay, T. W. H.; Thomas, H. E. Perforin and Fas Induced by IFNγ and TNFα Mediate Beta Cell Death by OT-I CTL. International Immunology 2006, 18, 837-846, doi:10.1093/intimm/dx1020.
19. Li, X.; Feng, Y.; Shang, F.; Yu, Z.; Wang, T.; Zhang, J.; Song, Z.; Wang, P.; Shi, B.; Wang, J. Characterization of the Therapeutic Effects of Novel Chimeric Antigen Receptor T Cells Targeting CD38 on Multiple Myeloma. Frontiers in Oncology 2021, 11, doi:10.3389/fonc.2021.703087.
20. Larson, R. C.; Kann, M. C.; Bailey, S. R.; Haradhvala, N. J.; Llopis, P. M.; Bouffard, A. A.; Scarfó, I.; Leick, M. B.; Grauwet, K.; Berger, T. R.; et al. CAR T Cell Killing Requires the IFNγR Pathway in Solid but Not Liquid Tumours. Nature 2022, 604, 563-570, doi:10.1038/s41586-022-04585-5.
21. Wang, X.; Berger, C.; Wong, C. L. W.; Forman, S. J.; Riddell, S. R.; Jensen, M. C. Engraftment of Human Central Memory-Derived Effector CD8+ T Cells in Immunodeficient Mice. Blood 2011, 117, 1888-1898, doi:10.1182/blood-2010-10-310599.
22. Weaver, J. C.; McGrath, P.; Adams, S. Gel Microdrop Technology for Rapid Isolation of Rare and High Producer Cells. Nature Medicine 1997, 3, 583-584, doi:10.1038/nm0597-583.
23. Kenney, J. S.; Gray, F.; Ancel, M. H.; Dunne, J. F. Production of Monoclonal Antibodies Using a Secretion Capture Report Web. Bio/Technology 1995, 13, 787-790, doi:10.1038/nbt0895-787.
24. Yokoyama, W. M.; Christensen, M.; Santos, G. D.; Miller, D.; Ho, J.; Wu, T.; Dziegelewski, M.; Neethling, F. A. Production of Monoclonal Antibodies. Current Protocols in Immunology 2013, 102, doi:10.1002/0471142735.im0205s102.
25. Vallurupalli, M.; Berliner, N. Emapalumab for the Treatment of Relapsed/Refractory Hemophagocytic Lympho-histiocytosis. Blood 2019, 134, 1783-1786, doi:10.1182/blood.2019002289.
26. Fujimoto, M.; Poe, J. C.; Inaoki, M.; Tedder, T. F. CD19 Regulates B Lymphocyte Responses to Transmembrane Signals. Seminars in Immunology 1998, 10, 267-277, doi:10.1006/smim.1998.9999.
27. Williams, A. F.; Barclay, A. N. The Immunoglobulin Superfamily—Domains for Cell Surface Recognition. Annual Review of Immunology 1988, 6, 381-405, doi:10.1146/annurev.iy.06.040188.002121.
28. Zhou, L. J.; Ord, D. C.; Hughes, A. L.; Tedder, T. F. Structure and Domain Organization of the CD19 Antigen of Human, Mouse, and Guinea Pig B Lymphocytes. Conservation of the Extensive Cytoplasmic Domain. The Journal of Immunology 1991, 147, 1424-1432, doi:10.4049/jimmunol.147.4.1424.

What is claimed is:

1. A fusion protein, comprising at least one single chain antibody fragment (scFV), and a cancer antigen or marker, or a fragment thereof,
wherein the scFV is capable of selectively binding to a cytokine released by chimeric antigen receptor (CAR) expressing cell,
and wherein the cancer antigen or marker comprises extracellular domain of CD19.

2. The fusion protein of claim 1, further comprising at least one or more linkers between scFV and the extracellular domain of CD19

3. The fusion protein of claim 1, wherein the cytokine released by chimeric antigen receptor (CAR) expressing cell comprises interferons, interferon gamma (IFN-γ), TNF-α,interleukins, IL1 IL2 IL6, IL10, IL12, IL15, IL17, IL18, IL21 IL35, growth factors, CSF, chemokines, lymphokines and monokines.

4. The fusion protein of claim 1, wherein the cytokine released by chimeric antigen receptor (CAR) expressing cell consists of interferon gamma (IFN-γ).

5. The fusion protein of claim 4, wherein the scFV comprises the amino acid sequence of SEQ ID NO:1.

6. The fusion protein of claim 4, wherein the fusion protein comprises the amino acid sequences of SEQ ID NO: 3 or SEQ ID NO:5.

7. The fusion protein of claim 2, wherein the linker comprises a flexible linker, a rigid linker, an enzymatically cleavable linker, an in vivo cleavable disulfide linker or a linker comprising protease-specific sequences.

8. The fusion protein of claim 7, wherein the linker comprises a proline rich linker, (Gly₄Ser)₃ (SEQ ID NO:15), (GGGS) (SEQ ID NO:16) or LEAGCKNFFPRSFTSCGSLE (SEQ ID NO: 11)).

9. The fusion protein of claim 1, wherein the cytokine released by chimeric antigen receptor (CAR) expressing cell consists of tumor necrosis factor alpha (TNF-α).

10. The fusion protein of claim 9, wherein the scFV comprises the amino acid sequence of SEQ ID NO: 13.

11. The fusion protein of claim 9, wherein the fusion protein comprises the nucleotide sequence of SEQ ID NO: 14.

12. A method for using the fusion protein of claim 1, comprising isolating or purifying a CAR expressing cell, wherein the scFV of the fusion protein binds with a cytokine secreted from a chimeric antigen receptor (CAR) expressing cell, and the extracellular domain of CD19 binds with an anti-CD19 chimeric antigen receptor (CAR).

13. The method of claim 12, wherein the cytokine secreted by the chimeric antigen receptor (CAR) expressing cell comprises interferons, interferon gamma (IFN-γ), TNF-α,interleukins, IL1 IL2 IL6, IL10, IL12, IL15, IL17, IL18, IL21 IL35, growth factors, CSF, chemokines, lymphokines or monokines.

14. The method of claim 13, wherein the secreted cytokine consists of interferon gamma (IFN-γ) or tumor necrosis factor alpha (TNF-α).

15. The method of claim 12, wherein isolation or purification of the CAR expressing cell is performed in a bed reaction packed with beads made of a polymer.

16. A fusion protein, comprising at least single chain antibody fragment (scFV), and a cancer antigen or marker, or a fragment thereof,
wherein the scFV is capable of selectively binding to a cytokine released by an immunotherapy cell.

17. The fusion protein of claim 16, wherein the immunotherapy cell comprises a chimeric antigen receptor (CAR) expressing cell.

18. A method of selecting CAR T cells from a polyclonal sample, comprising the fusion protein of claim 1.

19. A biomanufacturing process for producing T cells comprising the use of fusion proteins, wherein the fusion proteins are used to select T cells having predetermined characteristics,
wherein the fusion proteins comprise at least one single chain antibody fragment (scFV), and a cancer antigen or marker, or a fragment thereof,
wherein the scFV is capable of selectively binding to a cytokine released by chimeric antigen receptor (CAR) expressing cell.

20. The biomanufacturing process of claim 19, further comprising the use of beads in packed beds with functionalized surfaces that bind to CAR expressing cells.
